(19) United States
(12) Patent Application Publication
(10) Pub. No.: US 2002/0155084 A1
(43) Pub. Date: Oct. 24, 2002

(54) Nanoemulsion formulations

(75) Inventors: Blake J. Roessler, Ann Arbor, MI (US); James R. Baker JR., Ann Arbor, MI (US); Norman D. Weiner, Ann Arbor, MI (US); Ramachandran Chandrasekharan, Ypsilanti, MI (US); Huai Liang Wu, Lake Bluff, IL (US)

Correspondence Address: MEDLEN & CARROLL, LLP, 101 HOWARD STREET, SUITE 350, SAN FRANCISCO, CA, 94105, US

(73) Assignee: The Regents of the University of the Michigan, Ann Arbor, MI

(21) Appl. No.: 09/867,930

(22) Filed: May 30, 2001

Related U.S. Application Data
(60) Provisional application No. 60/208,726, filed on Jun. 2, 2000.

(51) Int. Cl. A61K 7/075

(52) U.S. Cl. 424/70.21

(57) ABSTRACT

The present invention relates to nanoemulsion formulations and methods for delivering biological agents to cells, and in particular nanoemulsion formulations for parenteral and non-parenteral delivery of biological agents to a subject. The nanoemulsion formulations of the present invention may be employed to permit the permeation of biological agents into the skin of subject or to increase the rate of permeation. Biological agents may be delivered by the nanoemulsion formulations of the present invention for diagnostic or therapeutic purposes.

This Application claims priority to Provisional Application No. 60/208,726 filed Jun. 2, 2000.

This invention was made with Government support under contract NIH NO1-AR-62226 and DARPA-DOD MDA972-97-1-0007. The government has certain rights in this invention.

### FIELD OF THE INVENTION

The present invention relates to nanoemulsion formulations and methods for delivering biological agents to cells, and in particular nanoemulsion formulations for parenteral and non-parenteral delivery of biological agents to a subject.

### BACKGROUND OF THE INVENTION

Advances in the field of biotechnology have led to significant advances in the treatment of diseases such as cancer, genetic diseases, arthritis and AIDS that were previously difficult to treat. Many such advances involve the administration of oligonucleotides and other nucleic acids to a subject, particularly human subjects. The administration of such molecules via parenteral routes has been shown to be effective for the treatment of certain diseases or disorders (See e.g., U.S. Pat. No. 5,595,978 and Roberton, Nature Biotechnology, 15:209 [1997])—both discussing antisense treatment of disease via parenteral modes of administration).

Non-parenteral routes of administration of oligonucleotides and other nucleic acids offers the promise of simpler, easier and less injurious administration of such nucleic acids without the need for sterile procedures and their concomitant expenses (e.g., hospitalization and/or physician fees). Liposomes possess many physical characteristics that make them attractive candidates as non-parenteral gene delivery vectors. However, liposomes are constructed from materials that are expensive and may require the use of potentially hazardous organic solvents, and usually require multi-step manufacturing process that yield small quantities of expensive, unstable vesicles with limited cargo capacity. Furthermore, another limitation of liposomal vectors is low ability to promote transgene expression when applied to follicular cells. Therefore, there is a need for compositions that are inexpensive, easy to manufacture, and that provide efficient levels of transgene expression in cells.

### SUMMARY OF THE INVENTION

The present invention relates to nanoemulsion formulations and methods for delivering biological agents to cells, and in particular nanoemulsion formulations for parenteral and non-parenteral delivery of biological agents to a subject. In some embodiments, the nanoemulsion formulations comprise an aqueous component, an oil component, and a surfactant mixture component. In certain embodiments, the nanoemulsion formulations do not contain short-chain alcohols. In other embodiments, the surfactant mixture component comprises a low hydrophilic-lipophilic balance (HLB) value surfactant and a high HLB value surfactant. In preferred embodiments, the nanoemulsion formulations further comprise a biological agent. In certain embodiments, the present invention provides methods for delivering biological agents to a subject employing the nanoemulsion formulations of the present invention.

In some embodiments, the present invention provides a composition comprising a nanoemulsion formulation, wherein the nanoemulsion formulation comprises an aqueous component, an oil component, and a surfactant mixture component, wherein the surfactant mixture component comprises two surfactants. In certain embodiments, the surfactant mixture component comprises two non-ionic surfactants. In some embodiments, the surfactant mixture component comprises propylene glycol monolaurate and sucrose monolaurate. In preferred embodiments, the surfactant mixture component comprises sorbitan monolaurate (Span 80) and POE (20) sorbitan monooleate (Tween 80).

In other embodiments, the surfactant mixture component comprises a low HLB value surfactant and a high HLB value surfactant. In other embodiments, the low and high HLB value surfactants are generally regarded as safe (GRAS) for animal (e.g. human) therapeutic applications. In preferred embodiments, the surfactant mixture component comprises a low HLB value non-ionic surfactant and a high HLB value non-ionic surfactant. In certain embodiments, the low HLB value surfactant has an HLB value between approximately 1.0 and 9.9 and the high HLB value surfactant has an HLB value between approximately 10.0 and 19.0. In preferred embodiments, the low HLB value surfactant has an HLB value between approximately 4.0 and 6.0 and the high HLB value surfactant has an HLB value between approximately 14.0 and 16.0. In preferred embodiments, the low HLB value surfactant has an HLB value between approximately 3.3 and 5.3 and the high HLB value surfactant has an HLB value between approximately 14.0 and 16.0. In certain embodiments, the low HLB value surfactant is selected from propylene glycol monostearate, glycerol monoleate, glycerol monostearate, acetylated monoglycerides (stearate), sorbitan monooleate (Span 80), propylene glycol monolaurate, sorbitan monostearate, and glycerol monolaurate; and the high HLB value surfactant is selected from POE (20) sorbitan monostearate, sucrose monolaurate, POE (20) sorbitan monooleate (Tween 80), and POE (20) sorbitan monopalmitate.

In other preferred embodiments, the surfactant mixture component of the nanoemulsion comprises a low HLB value surfactant with an HLB value between approximately 4.0 and 4.6 and a high HLB value surfactant with an HLB value between approximately 14.7 and 15.3. In particularly preferred embodiments, the low HLB value surfactant has an HLB value of approximately 4.3 and is selected from diethylene glycol monostearate, propylene glycol monolaurate, and sorbitan monooleate (span 80), and the high HLB value surfactant has an HLB value of approximately 15.0 and is selected from polyoxyethylene (20) sorbitan monostearate, sucrose monolaurate, POE (20) sorbitan monooleate (Tween 80), POE (16) lanolin alcohols, and acetylated POE (9) lanolin.

In some embodiments, the present invention provides a composition comprising a nanoemulsion formulation, wherein the nanoemulsion formulation comprises an aqueous component, an oil component, and a surfactant mixture component, wherein the surfactant mixture component comprises a low HLB value surfactant and a high HLB value surfactant. In certain embodiments, the ratio of low HLB value surfactant to the high HLB value surfactant in the surfactant mixture component is approximately 1:1 (e.g. ±5% of either surfactant). In other embodiments, the low HLB value surfactant is present in a greater amount than the high HLB value surfactant. In particular embodiments, the ratio of low HLB value surfactant to the high HLB value surfactant in the surfactant mixture component is approximately 5:3 (e.g. ±5% of either surfactant). In preferred embodiments, the ratio of low HLB value surfactant to the high HLB value surfactant in the surfactant mixture component is approximately 2:1 (e.g. ±5% of either surfactant). In particularly preferred embodiments, the ratio of low HLB value surfactant to the high HLB value surfactant in the surfactant mixture component is approximately 3:1 (e.g. ±5% of either surfactant). In other embodiments, the ratio of the low HLB value surfactant to the high HLB value surfactant is greater than 3:1. In further embodiments, the ratio of the low HLB value surfactant to the high HLB value surfactant is at least 3:1.

In some embodiments, the present invention provides a composition comprising a nanoemulsion formulation, wherein the nanoemulsion formulation comprises an aqueous component, an oil component, and a surfactant mixture component. In certain embodiments, the surfactant mixture component constitutes approximately 5-99% of the nanoemulsion formulation. In other embodiments, the surfactant mixture component constitutes approximately 10-45% of the nanoemulsion formulation. In preferred embodiments, the surfactant mixture component constitutes approximately 15-40% of the nanoemulsion formulation. In particularly preferred embodiments, the surfactant mixture component constitutes approximately 20-35% of the nanoemulsion formulation. In certain embodiments, the surfactant mixture component constitutes greater than 20% of the nanoemulsion formulation (e.g. 30%).

In some embodiments, the present invention provides a composition comprising a nanoemulsion formulation, wherein the nanoemulsion formulation comprises an aqueous component, an oil component, and a surfactant mixture component. In certain embodiments, the oil component is selected from soybean oil, avocado oil, squalene oil, olive oil, canola oil, corn oil, rapeseed oil, safflower oil, sunflower oil, fish oils, flavor oils, and mixtures thereof. In preferred embodiments, the oil component is selected from soybean oil and olive oil. In certain embodiments, the oil component constitutes approximately 0.1-95% of the nanoemulsion formulation. In other embodiments, the oil component constitutes approximately 40-90% of the nanoemulsion formulation. In preferred embodiments, the oil component constitutes approximately 50-80% of the nanoemulsion formulation. In particularly preferred embodiments, the oil component constitutes approximately 60-75% of the nanoemulsion formulation. In certain embodiments, the oil component constitutes greater than 60% of the nanoemulsion formulation.

In some embodiments, the present invention provides a composition comprising a nanoemulsion formulation, wherein the nanoemulsion formulation comprises an aqueous component, an oil component, and a surfactant mixture component. In certain embodiments, the aqueous component is selected from distilled water, deionized water, normal saline, phosphate buffered saline and mixtures thereof. In particular embodiments, aqueous component further comprises propylene glycol. In certain embodiments, the aqueous component constitutes approximately 0.1-35% of the nanoemulsion formulation. In other embodiments, the aqueous component constitutes approximately 1-20% of the nanoemulsion formulation. In preferred embodiments, the aqueous component constitutes approximately 2-10% of the nanoemulsion formulation. In particularly preferred embodiments, the aqueous component constitutes approximately 2-6% of the nanoemulsion formulation. In certain embodiments, the aqueous component constitutes less than 6% of the nanoemulsion formulation.

In further embodiments, the present invention provides a composition comprising a nanoemulsion formulation, wherein the nanoemulsion formulation comprises an aqueous component, an oil component, a biological agent, and a surfactant mixture component. In certain embodiments, the biological agent is a drug (e.g., antimalarial agents, anti-neoplastic agents, antihistamines, biogenic amines, antidepressants, anticholinergics, antiarrhythimics, antiemetics, antibiotics and analgesics). In certain embodiments, the biological agent is present in therapeutically effective amounts. In other embodiments, the biological agents is a peptide (e.g., recombinant protein). In different embodiments, the biological agent is a carbohydrate or lipid. In other embodiments, the biological agent is an antimicrobial. In further embodiments, the biological agent is nucleic acid. In further embodiments, the biological agent is a nucleic acid selected from DNA, cDNA, RNA (full length mRNA, ribozymes, antisense RNA, and decoys), oligodeoxynucleotides (phosphodiesters, phosphothioates, phosphoramidites, and all other chemical modifications), oligonucleotide, linear and closed circular plasmid DNA, and other expression vectors. In preferred embodiments, the biological agent is an expression plasmid. In certain embodiments, the expression plasmid is present in therapeutically effective amounts. In some embodiments, the expression plasmid expresses recombinant peptide in cells. In other embodiments, the expression plasmid expresses RNA transcripts in cells (e.g., antisense RNA).

In some embodiments, the present invention provides a composition comprising a nanoemulsion formulation that permits an expression vector (e.g., plasmid) to express a recombinant peptide at a mean level of at least 57.0 pg/cm² in cells. In other embodiments, the nanoemulsion formulation permits an expression vector to express a recombinant peptide at a mean level of at least 100.0 pg/cm² in cells. In other embodiments, the nanoemulsion formulations permits an expression vector to express a recombinant peptide at a mean level of at least 285.0 pg/cm² in cells, or at a mean level of at least 376.0 pg/cm² in cells. In other embodiments, the cells are the skin cells of a subject. In certain embodiments, the recombinant peptide expressed by the expression vector is human interferon-α2.

In some embodiments, the present invention provides a composition comprising a nanoemulsion formulation that permits an expression vector (e.g., plasmid) to express RNA transcripts at a level of at least 5.0×10⁴ transcripts/cm² in cells. In other embodiments, the nanoemulsion formulation allows an expression vector to express RNA transcripts at a level of at least 5.0×10⁵ transcripts/cm² in cells. In other embodiments, the cells of the subject are skin cells. In some embodiments, the RNA transcript expressed by the expression vector is antisense RNA. In further embodiments, the nanoemulsion formulation does not contain short-chain alcohols.

In some embodiments, the present invention provides a composition comprising a nanoemulsion formulation that permits a skin permeation rate of at least 0.447% per hour for a biological agent in said nanoemulsion formulation. In other embodiments, the nanoemulsion formulation permits a skin permeation rate of at least 0.519% per hour for a biological agent in said nanoemulsion. In a different embodiment, the nanoemulsion formulation permits a skin permeation rate of at least 0.615% per hour for a biological agent in the nanoemulsion formulation. In preferred embodiments, the nanoemulsion formulation permits a skin permeation rate of at least 0.823% per hour for a biological agent in the nanoemulsion formulation. In particularly preferred embodiments, the nanoemulsion formulation permits a skin permeation rate of approximately 0.870% per hour for a biological agent in the nanoemulsion formulation. In certain embodiments, the nanoemulsion formulation comprises a biological agent. In certain embodiments, the biological agent is a protein, carbohydrate, lipid, nucleic acid, or mixture thereof. In further embodiments, the nanoemulsion formulation does not contain short-chain alcohols.

In other embodiments, the present invention provides a method comprising; a) providing; i) a nanoemulsion formulation, wherein the nanoemulsion formulation comprises a biological agent, and ii) a subject; and b) administering the nanoemulsion formulation to the subject. In other embodiments, the present invention provides a method comprising; a) providing; i) a nanoemulsion formulation, wherein the nanoemulsion formulation comprises a biological agent, and ii) a subject comprising skin; and b) administering the nanoemulsion formulation to the skin of the subject such that the biological agent has a permeation rate of at least 0.447% per hour. In certain embodiments, the biological agent is present in therapeutically effective amounts. In other embodiments, the present invention provides a method comprising; a) providing; i) a nanoemulsion formulation, wherein the nanoemulsion formulation comprises a biological agent, and ii) a subject comprising skin; and b) administering the nanoemulsion formulation to the skin of the subject such that the biological agent has a permeation rate selected from at least 0.5197% per hour, at least 0.615% per hour, at least 0.823% per hour, or approximately 0.870% per hour. In certain embodiments, the biological agent is a protein, carbohydrate, lipid, nucleic acid, or mixture thereof. In further embodiments, the nanoemulsion formulation does not contain short-chain alcohols.

In other embodiments, the present invention provides a method comprising; a) providing; i) a nanoemulsion formulation, wherein the nanoemulsion formulation comprises an expression vector, and ii) a subject; and b) administering the nanoemulsion formulation to the subject. In certain embodiments, the expression vector is present in therapeutically effective amounts. In other embodiments, the present invention provides a method comprising; a) providing; i) a nanoemulsion formulation, wherein the nanoemulsion formulation comprises an expression vector, and ii) a subject comprising skin cells; and b) administering the nanoemulsion formulation to the skin cells of the subject such that the expression vector expresses RNA transcripts at a level of at least 5.0×10⁴ transcripts/cm² in the skin of the subject. In other embodiments, the present invention provides a method comprising; a) providing; i) a nanoemulsion formulation, wherein the nanoemulsion formulation comprises an expression vector, and ii) a subject comprising skin cells; and b) administering the nanoemulsion formulation to the skin cells of the subject such that the expression vector expresses RNA transcripts at a level of at least 5.0×10⁵ transcripts/cm² in the skin of the subject. In some embodiments, the RNA transcript expressed by the expression vector is antisense RNA. In further embodiments, the nanoemulsion formulation does not contain short-chain alcohols.

In other embodiments, the present invention provides a method comprising; a) providing; i) a nanoemulsion formulation, wherein the nanoemulsion formulation comprises a biological agent, and ii) a subject; and b) administering the nanoemulsion formulation to the subject. In some embodiments, the nanoemulsion formulation is administered to the subject parenterally. In further embodiments, the nanoemulsion formulation is administered to the subject in a mode selected from: intravenously, intra-muscularly, subcutaneously, intradermally, intraperitoneally, intrapleurally, and intrathecally. In preferred embodiments, the nanoemulsion formulation is administered to the subject non-parenterally. In further embodiments, the nanoemulsion formulation is administered to the subject in a mode selected from: buccal, sublingual, endoscopic, oral, rectal, transdermal, nasal, intratracheal, pulmonary, urethral, vaginal, ocular, and topical. A preferred mode of administration is topically to the skin of a subject. In certain embodiments, the biological agent is present in therapeutically effective amounts. In preferred embodiments, the nanoemulsion formulations of the present invention are non-irritating when applied to the skin of a subject.

In other embodiments, the present invention provides a method comprising; a) providing; i) a nanoemulsion formulation, wherein the nanoemulsion formulation comprises a biological agent, and ii) a skin sample; and b) administering the nanoemulsion formulation to the skin sample. In other embodiments, the present invention provides a method comprising; a) providing; i) a nanoemulsion formulation, wherein the nanoemulsion formulation comprises a biological agent, and ii) a skin sample; and b) administering the nanoemulsion formulation to the skin sample such that the biological agent has a permeation rate of at least 0.447% per hour. In other embodiments, the present invention provides a method comprising; a) providing; i) a nanoemulsion formulation, wherein the nanoemulsion formulation comprises a biological agent, and ii) a skin sample; and b) administering the nanoemulsion formulation to the skin sample such that the biological agent has a permeation rate selected from at least 0.5197% per hour, at least 0.615% per hour, at least 0.823% per hour, or approximately 0.870% per hour. In certain embodiments, the biological agent is a protein, carbohydrate, lipid, nucleic acid, or mixture thereof. In further embodiments, the nanoemulsion formulation does not contain short-chain alcohols.

In other embodiments, the present invention provides a method comprising; a) providing; i) a nanoemulsion formulation, wherein the nanoemulsion formulation comprises an expression vector, and ii) a skin sample; and b) administering the nanoemulsion formulation to the skin sample. In other embodiments, the present invention provides a method comprising; a) providing; i) a nanoemulsion formulation, wherein the nanoemulsion formulation comprises an expression vector, and ii) a skin sample; and b) administering the nanoemulsion formulation to the skin sample such that the expression vector expresses RNA transcripts at a level of at least 5.0×10⁴ transcripts/cm² in the skin sample. In other embodiments, the present invention provides a method comprising; a) providing; i) a nanoemulsion formulation, wherein the nanoemulsion formulation comprises an expression vector, and ii) a skin sample; and b) administering the nanoemulsion formulation to the skin sample such that the expression vector expresses RNA transcripts at a level of at least 5.0×10⁵ transcripts/cm² in the skin sample. In some embodiments, the RNA transcript expressed by the expression vector is antisense RNA. In further embodiments, the nanoemulsion formulation does not contain short-chain alcohols. In other embodiments, the nanoemulsion formulations do not contain methanol, ethanol, propanol, butanol, pentanol, or hexanol, or any combination thereof.

### DESCRIPTION OF THE FIGURES

FIG. 1 shows the mean particle sizes for 5:3 Span 80:Tween 80 nanoemulsions of the present invention. FIG. 1A shows the nanoemulsion without DNA (mean particle size of 42.3+/−14.6 nm), and FIG. 1B shows the nanoemulsion with plasmid DNA (mean particle size of 32.1+/−20.2 nm).

FIG. 2 shows the relative time course of transgene expression following application of a single dose of aqueous DNA or nanoemulsion DNA.

FIG. 3 shows a comparison between the level of transgene expression produced by nanoemulsion formulations containing a huInfα2 plasmid in normal murine skin (C57 BL/6J) and hairless murine skin.

FIG. 4 shows a pseudo-ternary phase diagram of a 1:1 Span 80:Tween 80 nanoemulsion formulation with optically isotropic regions at low water content designated as L₂.

FIG. 5 shows a pseudo-ternary phase diagram of a 2:1 Span 80:Tween 80 nanoemulsion formulation with optically isotropic regions at low water content designated as L₂.

FIG. 6 shows a pseudo-ternary phase diagram of a 3:1 Span 80:Tween 80 nanoemulsion formulation with optically isotropic regions at low water content designated as L₂.

FIG. 7 shows the results of permeation of inulin across hairy and hairless mouse and hairy rat skin following topical in vitro application of an nanoemulsion formulation with 5 μg/ml inulin (2:1 Span 80-Tween 80 nanoemulsion).

FIG. 8 shows the permeation profiles of inulin across hairy mouse skin following topical in vitro application of 2:1 Span 80-Tween 80 nanoemulsion formulations with widely differing concentrations of inulin (0.80 mg/ml and 5 μg/ml).

FIG. 9 shows the permeation profiles of 2:1 Span 80-Tween 80 nanoemulsions with two different markers of divergent molecular weight: inulin (MW=5,000 Da) and tranexamic acid (MW=157.2 Da).

FIG. 10 shows the permeation of inulin and tranexamic acid in 5% sodium lauryl sulfate (SLS), or with water.

FIG. 11 presents profiles of inulin permeation across the hairy murine skin following topical application of the various nanoemulsion formulations (listed in Table 3).

FIG. 12 shows the correlation between permeation rate of inulin and percent Tween 80 following topical in vitro application of a variety of formulations to hairy mouse skin.

FIG. 13 is a comparison of the profiles from nanoemulsion formulations of identical total surfactant mixture component, oil component, and aqueous component.

FIG. 14 shows a comparison of profiles from nanoemulsion formulations, with the same amount of each component, but with an aqueous phases containing 50/50 (v/v) mixture of propylene glycol and isotonic HEPES buffer, pH 7.4.

FIG. 15 shows the average permeation rates of inulin from the nanoemulsion formulations listed in Table 5 plotted as a function of Tween 80 content in the formulation.

### DEFINITIONS

To facilitate an understanding of the present invention, a number of terms and phrases are defined below:

As used herein, “short chain alcohols” are those alcohol molecules that have six or fewer carbon atoms including, but not limited to, butanol, pentanol, and hexanol.

As used herein, the term “skin sample” refers to both a portion of the living skin tissue on a subject (host) and skin tissue that has been separated from the originating subject or host (e.g. in vitro culture samples, primary culture samples, and ex vivo skin samples (See e.g. Example 3).

As used herein, the term “biological agent” refers to any molecule, compound, or composition that is capable of producing a therapeutic or diagnostic affect in a subject, skin sample, or cell. In one embodiment, biological agent(s), when present in an effective amount, react with and/or effect (e.g. alter) living cells and organisms. Examples of biological agents include, but are not limited to; expression plasmids, proteins, vitamins, steroids, antisense RNA, cytokines, enzymes, vaccines, anti-neoplastic agents, drugs, and antibiotics.

As used herein, the term “antisense” is used in reference to RNA sequences that are complementary to a specific RNA sequence (e.g., mRNA). Included within this definition are antisense RNA (“asRNA”) molecules involved in gene regulation by bacteria. Antisense RNA may be produced by any method, including synthesis by splicing the gene(s) of interest in a reverse orientation to a viral promoter that permits the synthesis of a coding strand. Once introduced into an embryo, this transcribed strand combines with natural mRNA produced by the embryo to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

As used herein, the term “aqueous component” refers to the component of a composition that contains water (or is soluble in water). Where water is used, it may or may not contain salt(s) and may or may not be buffered. Thus, a variety of such components are contemplated including, but not limited to, distilled water, deionized water, normal saline, and phosphate buffered saline.

As used herein, the term “oil component” refers to any water immiscible component that is conventionally referred to as an oil. Examples, include, but are not limited to, soybean oil, avocado oil, squalene oil, olive oil, canola oil, corn oil, rapeseed oil, safflower oil, sunflower oil, fish oils, flavor oils, and mixtures thereof.

As used herein, the term “surfactant mixture component” refers to a mixture of two or more surfactants. Examples include, but are not limited to, a mixture of sorbitan monolaurate (Span 80) and POE (20) sorbitan monooleate (Tween 80), or a mixture of propylene glycol monolaurate and sucrose monolaurate.

As used herein, the term “nanoemulsion formulation” refers to a composition comprising an aqueous component, an oil component, and a surfactant mixture component.

As used herein, the term “non-parenteral administration” refers to modes of administration including, but not limited to, to the contacting, directly or otherwise, to all or a portion of the alimentary canal, skin, eyes, pulmonary tract, urethra, cervix or vagina of an animal. Examples of non-parenteral administration, include, but are not limited to, buccal, sublingual, endoscopic, oral, rectal, transdermal, topical, nasal, intratracheal, pulmonary, urethral, vaginal, and ocular administration.

As used herein, the term “parenteral administration” refers routes of administration other than to the alimentary canal or topical application to the skin, eyes, pulmonary tract, urethra, cervix or vagina. Examples of parenteral administration include, but are not limited to, intravenous, subcutaneous, and intramuscular.

The terms “pharmaceutically acceptable” or “pharmacologically acceptable,” as used herein, refer to compositions that do not produce adverse, allergic or other untoward reactions when administered to an animal or a human. As used herein, “pharmaceutically acceptable carrier” includes any and all solvents, dispersion media, coatings, isotonic and absorption delaying agents and the like.

As used herein, the term “subject” refers to any animal (e.g., warm blooded mammal), including, but not limited to, humans, non-human primates, rodents, farm animals (e.g., cattle, horses, pigs, goats, and sheep) and the like, that is to be the recipient of a particular treatment. The terms “subject” and “patient” are used interchangeably.

As used herein, the term “carrier” refers to a diluent, adjuvant, excipient, or vehicle with which a compound is administered. Such pharmaceutical carriers can be sterile liquids, such as water and oils, including those of petroleum, animal, vegetable, or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

As used herein, the term “therapeutically effective amount” is a functional term referring to an amount of material needed to make a qualitative or quantitative change in a clinically measured parameter for a particular subject. For example, prior to administration, the subject may exhibit measurable symptoms of disease (e.g. viral antigen load, clotting time, serum analyte level, vitamin or nutrient deficiency, etc), however, upon administration of a therapeutically effective amount the measurable symptom is found to have changed.

As used herein, the term “nucleic acid molecule” refers to any nucleic acid containing molecule including, but not limited to DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb or more on either end such that the gene corresponds to the length of the full-length mRNA. The sequences that are located 5′ of the coding region and that are present on the mRNA are referred to as 5′ non-translated sequences. The sequences that are located 3′ or downstream of the coding region and that are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

As used herein, the term “oligonucleotide,” refers to a short length of single-stranded polynucleotide chain. Oligonucleotides are typically less than 100 residues long (e.g., between 15 and 50), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

The term “recombinant protein” or “recombinant peptide” as used herein refers to a protein molecule or peptide molecule that is expressed from a recombinant DNA molecule.

The term “native protein” as used herein to indicate that a protein does not contain non-natural amino acid residues encoded by vector sequences; that is the native protein contains only those amino acids found in the protein as it occurs in nature. A native protein may be produced by recombinant means or may be isolated from a naturally occurring source.

The term “transgene” as used herein refers to any nucleic acid (e.g., gene sequence) that is introduced into the genome of an animal by experimental manipulations and may include gene sequences found in that animal so long as the introduced gene does not reside in the same location as does the naturally-occurring gene.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

As used herein, the term “hydrophilic-lipophilic balance” or “HLB” refers to a common way to classify surfactants. HLB is calculated: HLB=20(1−(S/A)), where S is the saponification number of the ester and A is the acid number of the resulting acid.

As used herein, the abbreviation “POE” stands for polyoxyethylene.

As used herein, the term “low HLB value surfactant” refers to those surfactants that have a HLB value of approximately 9.9 or less, preferably from approximately 1.0-9.9 (See, e.g. column 1 of Table 1).

As used herein, the term “high HLB value surfactant” refers to those surfactants that have an HLB value greater than approximately 9.9, preferably from approximately 10.0-19.0 (See e.g., column 2 of Table 1).

As used herein the term “skin permeation rate” refers to the percent of a biological agent (e.g. marker) able to penetrate a skin sample per hour as measured according to a standard Franz Diffusion Cell assay (See, Joel L. Zatz, Skin Permeation: fundamental and application, Allured Pub. Co., Wheaton, Ill., 1993, Chapter 4 ‘In vitro Methods for Measuring Skin Permeation’, pgs 93-111, and see Example 5).

### DESCRIPTION OF THE INVENTION

The present invention relates to nanoemulsion formulations and methods for delivering biological agents to cells, and in particular nanoemulsion formulations for parenteral and non-parenteral delivery of biological agents to a subject. In some embodiments, the nanoemulsion formulations comprise an aqueous component, an oil component, and a surfactant mixture component. In certain embodiments, the surfactant mixture component comprises a low HLB value surfactant and a high HLB value surfactant. In preferred embodiments, the nanoemulsion formulations further comprise a biological agent. In certain embodiments, the present invention provides methods of delivering biological agents to a subject employing the nanoemulsion formulations of the present invention.

I. Nanoemulsions

A nanoemulsion may be defined as transparent, liquid and isotropic dispersions composed of water, oil and surfactants that are thermodynamically stable. At precise compositions of ingredients, their formation is spontaneous and high shear energies are not required for their preparation. Nanoemulsions are typically prepared by first dispersing an oil in an aqueous surfactant solution and then adding a sufficient amount of a fourth component, generally a short chain-length alcohol (e.g., butanol, pentanol, or hexanol) to form a transparent system.

A nanoemulsion may be characterized as a water-in-oil nanoemulsion or an oil-in-water nanoemulsion. This characterization depends on the properties of the oil and surfactant used and on the structure and geometric packing of the polar heads and hydrocarbon tails of the surfactant molecules. Compared to conventional emulsions, nanoemulsions offer the advantage of solubilizing water-insoluble drugs in a formulation of thermodynamically stable droplets that are formed spontaneously.

However, although nanoemulsions possess several advantages with regards to their ease of preparation, high stability and clarity, (as noted above) the most commonly examined systems include short chain alcohols (as well as non-polar phases such as hexane) that make them unsuitable for most pharmaceutical purposes. Furthermore, when considering nanoemulsion formulations with nucleic acids, it is desirable to employ alcohol-free systems in order to avoid flocculation problems.

The nanoemulsion formulations of the present invention, in some embodiments, are short-chain alcohol-free nanoemulsion formulations that are prepared with oils and surfactants that are safe, non-toxic, non-irritating, and with components that are generally considered safe (GRAS). As such, the nanoemulsions of the present invention provide nanoemulsions that are safe for pharmaceutical use (e.g., topical delivery) and that may incorporate nucleic acids (e.g., plasmids) without flocculation problems.

In certain embodiments, the oil component of the nanoemulsion formulations of the present invention is present in an amount from about 0.1 to 95% by volume of the total nanoemulsion formulation. In other embodiments, the oil component comprises about 40 to 90%, preferably about 50 to 80%, more preferably about 60 to 75% by volume of the total nanoemulsion formulation. Suitable oils include, but are not limited to, soybean oil, avocado oil, squalene oil, olive oil, canola oil, corn oil, rapeseed oil, safflower oil, sunflower oil, fish oils, flavor oils, and mixtures thereof. Preferred oils are olive oil and soybean oil.

In certain embodiments, the aqueous component of the nanoemulsion formulations of the present invention is present in an amount from about 0.1-35% by volume of the total nanoemulsion formulation. In certain embodiments, the aqueous component comprises about 1.0-20%, preferably 2-10%, more preferably about 2-6% by volume of the total nanoemulsion formulation. Examples of aqueous components include, but are not limited to, distilled water, deionized water, normal saline, and phosphate buffered saline. In certain embodiments, the aqueous component further comprises propylene glycol. In some embodiments, the presence of propylene glycol in the aqueous component permits the amount of aqueous component to be increased. In this regard, in certain embodiments, propylene glycol is added to the aqueous phase to increase the percent of aqueous component, thus allowing a greater amount of biological agents to be added to the aqueous component.

In some embodiments, the surfactant mixture component of the nanoemulsion formulations comprises a mixture of two or more surfactants. In some embodiments, the surfactant mixture component of the nanoemulsion formulations of the present invention is present in an amount from about 5-100% by volume of the total nanoemulsion. In preferred embodiments, the surfactant mixture component is present in about 10-45%, preferably about 15-40%, more preferably about 20-35% by volume of the total nanoemulsion formulation.

The most common way for classifying surfactants, both natural and synthetic, is by the use of the hydrophilic-lipophilic balance (HLB) value. The nature of the hydrophilic group (also known as the “head”) provides the most useful means for categorizing the different surfactants used in formulations (Rieger, in “Pharmaceutical Dosage Forms: Disperse Systems, Vol. 1, Lieberman, Rieger and Banker, Eds., Marcel Dekker, Inc., New York, N.Y., 1988, p. 285). HLB is calculated as follows; HLB=20(1−(S/A)), where S is the saponification number of the ester and A is the acid number of the resulting acid.

If the surfactant molecule is not ionized, it is classified as a nonionic surfactant. Nonionic surfactants find wide application in pharmaceutical and cosmetic products and are usable over a wide range of pH values. In general, their HLB values range from 1 to about 18 depending on their structure. Table 1 lists some exemplary non-ionic surfactants (with both ‘low’ and ‘high’ HLB values) useful in the present invention. Any surfactant that allows the oil phase to remain suspended in the water phase can be used. Nonionic surfactants have advantages over ionic emulsifiers because they are compatible with a broad pH range and often form more stable emulsions than do ionic (e.g., soap-type) emulsifiers.

TABLE 1
Non-Ionic Surfactants with Hydrophile/Lipophile
Balance (HLB) values

| Low Non-Ionic Surfactants | HLB | High Non-Ionic Surfactants | HLB |
|---|---|---|---|
| Oleic acid | 1.0 | POE (5) sorbitan monooleate | 10.0 |
| Lanolin alcohols | 1.0 | POE (40) sorbitol hexaoleate | 10.2 |
| Acetylated sucrose diester | 1.0 | PEG 400 dilaurate | 10.4 |
| Ethylene glycol distearate | 1.3 | POE (5) nonylphenol | 10.5 |
| Acetylated monoglycerides | 1.5 | POE (20) sorbitan tristearate | 10.5 |
| Sorbitan trioleate | 1.8 | POP/POE condensate | 10.6 |
| Glycerol dioleate | 1.8 | POE (6) nonylphenol | 10.9 |
| Sorbitan tristearate | 2.1 | POE (20) lanolin (ether and ester) | 11.0 |
| Ethylene glycol monostearate | 2.9 | POE (20) sorbitan trioleate | 11.0 |
| Sucrose distearate | 3.0 | POE (8) stearic acid (monoester) | 11.1 |
| Decaglycerol decaoleate | 3.0 | POE (50) sorbitol hexaoleate | 11.4 |
| Propylene glycol monostearate | 3.4 | POE (6) tridecyl alcohol | 11.4 |
| Glycerol monoleate | 3.4 | PEG 400 monostearate | 11.7 |
| Diglycerine sesquioleate | 3.5 | POE (8) nonylphenol | 12.3 |
| Sorbitan sesquioleate | 3.7 | POE (10) stearyl alcohol | 12.4 |
| Glycerol monostearate | 3.8 | POE (8) tridecyl alcohol | 12.7 |
| Acetylated monoglycerides (stearate) | 3.8 | POE (8) lauric acid (monester) | 12.8 |
| Decaglycerol octaoleate | 4.0 | POE (10) cetyl alcohol | 12.9 |
| Diethylene glycol monostearate | 4.3 | Acetylated POE (10) lanolin | 13.0 |
| Sorbitan monooleate (Span 80) | 4.3 | POE (20) glycerol monostearate | 13.1 |
| Propylene glycol monolaurate | 4.5 | PEG 400 monolaurate | 13.1 |
| POE (1.5) nonyl phenol | 4.6 | POE (16) lanolin alcohol | 13.2 |
| Sorbitan monostearate | 4.7 | POE (4) sorbitan monolaurate | 13.3 |
| POE (2) oleyl alcohol | 4.9 | POE (10) nonylphenol | 13.3 |
| POE (2) stearyl alcohol | 4.9 | POE (15) tall oil fatty acids (ester) | 13.4 |
| POE sorbital beeswax derivative | 5.0 | POE (10) octylphenol | 13.6 |
| PEG 200 distearate | 5.0 | PEG 600 monosteatate | 13.6 |
| Glycerol monolaurate | 5.2 | POE (24) cholesterol | 14.0 |
| POE (2) octyl alcohol | 5.3 | POE (14) nonylphenol | 14.4 |
| Decaglycerol tetraoleate | 6.0 | POE (12) lauryl alcohol | 14.5 |
| PEG 300 dilaurate | 6.3 | POE (20) sorbitan monostearate | 14.9 |
| Sorbitan monopalmitate | 6.7 | Sucrose monolaurate | 15.0 |
| N,N,-Dimethylstearamide | 7.0 | POE (20) sorbitan monooleate (Tween 80) | 15.0 |
| PEG 400 distearate | 7.2 | POE (16) lanolin alcohols | 15.0 |
| POE (5) lanolin alcohol | 7.7 | Acetylated POE (9) lanolin | 15.0 |
| PEG ether of linear alcohol | 7.7 | POE (20) stearyl alcohol | 15.3 |
| POE octylphenol | 7 8 | POE (20) oleyl alcohol | 15.3 |
| Soya lecithin | 8.0 | PEG 1000 monooleate | 15.4 |
| Diacetylated tartaric acid esters | 8.0 | POE (20) sorbitan monopalmitate | 15.6 |
| POE (4) stearic acid (monoester) | 8.0 | POE (20) cetyl alcohol | 15.7 |
| Sorbitan monolaurate | 8.6 | POE (25) propylene glycol monostearate | 16.0 |
| POE (4) nonylphenol | 8.9 | POE (20) nonylphenol | 16.0 |
| Isopropyl ester of lanolin fatty acids | 9.0 | PEG 1000 monolaurate | 16.5 |
| POE (4) tridecyl alcohol | 9.3 | POE (20) sorbitan monolaurate | 16.9 |
| POE (4) lauryl alcohol | 9.5 | POE (23) lauryl alcohol | 16.9 |
| | | POE (40) stearic acid (monoester) | 16.9 |
| | | POE (50) lanolin (ether and ester) | 17 0 |
| | | POE (25) soyasterol | 17.0 |
| | | POE (30) nonylphenol | 17.1 |
| | | PEG 4000 distearate | 17.3 |
| | | POE (50) stearic acid (monoester) | 17.9 |
| | | Sodium Oleate | 18.0 |
| | | POE (70) dinonylphenol | 18.0 |
| | | POE (20) castor oil (ether, ester) | 18.1 |

POE = polyoxyethylene
PEG = poly (ethylene glycol)
POP = polyoxypropylene

If the surfactant molecule carries a negative charge when it is dissolved or dispersed in water, the surfactant is classified as anionic. Anionic surfactants include, but are not limited to, carboxylates such as soaps, acyl lactylates, actyl amides of amino acids, esters of sulfuric acid such as alkyl sulfates and ethoxylated alkyl sulfates, sulfonates such as alkyl benzene sulfonates, acyl isethionates, acyl taurates sulfosuccinates, and phosphates. Further examples with associated HLB values include, but are not limited to, calcium stearoxyl-2-lactylate (5.1), sodium-o-stearyllactate (5.7), sodium stearoyllactylate (8.3), calcium dodecyl benzene sulfonate (9.0), glycerol monostearate (11.0), alkyl aryl sulfonate (11.7), triethanolamine oleate soap (12.0), and potassium oleate (20.0).

If the surfactant molecule carries a positive charge when it is dissolved or dispersed in water, the surfactant is classified as cationic. Cationic surfactants include, but are not limited to, quaternary ammonium salts and ethoxylated amines. Further examples with associated HLB values include, but are not limited to, high molecular-weight fatty amine blends (4.5), high molecular weight amine blends (7.5), tertiary amines: POE fatty amines (13.9), and POE (20) tallow amine (15.5).

If the surfactant molecule has the ability to carry either a positive or negative charge, the surfactant is classified as amphoteric. Amphoteric surfactants include, but are not limited to, acrylic acid derivatives, substituted alkylamides, N-alkylbetaines and phosphatides.

In certain embodiments, the surfactants employed in the surfactant mixture component are generally regarded as safe (GRAS) surfactants (See, e.g, Osborne et al., Pharmaceutical Technology, pgs 58-60, [November 1997]). In some embodiments, the surfactant mixtures of the present invention comprise a least one surfactant with an HLB value of approximately 1.0-9.9 (e.g. a low HLB non-ionic surfactant, See column 1 of Table 1), and at least one surfactant with an HLB value of greater than approximately 9.9 (e.g. a high HLB non-ionic surfactant, See column 2 of Table 1). Preferred low HLB surfactants have HLB values in the range of approximately 3.3-5.3 (e.g. ±0.3). Preferred high HLB surfactants have HLB values in the range of approximately 14.0-16.0 (e.g. ±0.3). Examples of preferred low HLB non-ionic surfactants, include, but are not limited to; propylene glycol monostearate, glycerol monoleate, glycerol monostearate, acetylated monoglycerides (stearate), sorbitan monooleate, propylene glycol monolaurate, sorbitan monostearate, and glycerol monolaurate. A particularly preferred low HLB non-ionic surfactant is sorbitan monooleate (Span 80). Examples of preferred high HLB non-ionic surfactants include, but are not limited to; POE (20) sorbitan monostearate, sucrose monolaurate, POE (20) sorbitan monooleate, and POE (20) sorbitan monopalmitate. A particularly preferred high HLB non-ionic surfactant is polyoxyethylene (20) sorbitan monooleate (Tween 80).

In certain embodiments, the ratio of the low HLB non-ionic surfactant to the high HLB non-ionic surfactant that comprise the surfactant mixture is approximately 1:1 (e.g. ±5% of either surfactant). In other embodiments, the ratio of low HLB non-ionic surfactant to the high HLB non-ionic surfactant is approximately 5:3 (e.g. ±5% of either surfactant), preferably 2:1 (e.g. ±5% of either surfactant), more preferably approximately 3:1 (e.g. ±5% of either surfactant). In other embodiments, the ratio of the low HLB value surfactant to the high HLB value surfactant is greater than 3:1. Other ratios are also contemplated and may be determined, for example, by employing phase diagram methodology, as described below. Generally, the high HLB non-ionic surfactant is present in the same percent or less than the low HLB non-ionic surfactant.

The ratio of low to high HLB non-ionic surfactants, the types of non-ionic surfactants selected, and the total percent that the surfactant mixture component affect the amount of aqueous component and oil component needed to make a nanoemulsion. Likewise, the percent and type of oil component or aqueous component affect the amount of surfactant mixture component needed to make a nanoemulsion. The appropriate concentrations of each reagent needed to make a nanoemulsion can be evaluated by employing, for example, phase diagram methodology.

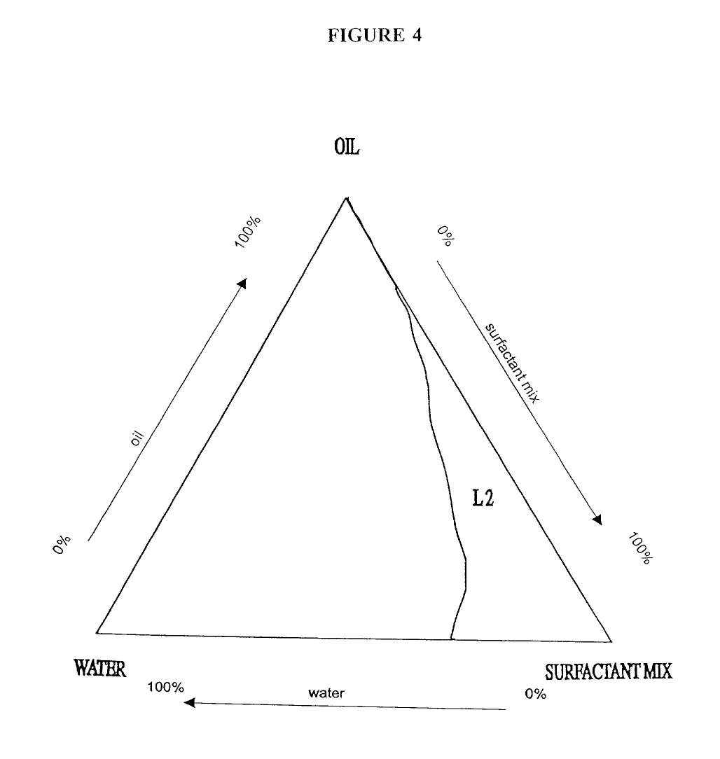

The phenomenological approach utilizing phase diagrams has been extensively studied and has yielded a comprehensive knowledge of how to formulate nanoemulsions (See e.g., Rosoff, in Pharmaceutical Dosage Forms: Disperse Systems, Vol. 1, Lieberman, Riegar and Banker, Eds., Marcel Dekker, Inc., New York, N.Y., 1988, p. 245). Phase diagram methodology was employed in Example 4 (See, FIGS. 4-6) to determine the appropriate concentrations of the aqueous component and oil component needed to form nanoemulsions for 1:1, 2:1, and 3:1 ratios of Span 80-Tween 80. This type of procedure is employed, for example, when selecting other ratios of Span 80-Tween 80, or when selecting other types of non-ionic surfactant mixture components, and when selecting various concentrations of the all the nanoemulsion components.

The nanoemulsion formulations of the present invention, in some embodiments, are further combined with biological agents. In some embodiments, the nanoemulsion is made by first mixing the surfactant mixture component (e.g., a low HLB non-ionic surfactant and a high non-ionic surfactant) with the oil component. In certain embodiments, the surfactant mixture component/oil component formulation is then warmed (e.g., 50° C.) and sterile filtered with a microfilter. In other embodiments, the surfactant mixture component/oil component formulation is only heated, or only filtered, or neither. Next, the aqueous phase (with or without biological agent(s) present) is added to the surfactant mixture component/oil component formulation and mixed gently to yield a clear water-in-oil nanoemulsion (which may comprise a biological agent).

The ability of any nanoemulsion formulations comprising a biological agent to deliver biological agents locally to a subject may be tested, for example, in murine skin assays (both permeation rates and ability to deliver and/or express expression vectors). For example, the ability of the nanoemulsion formulations of the present invention comprising an expression vector (e.g. plasmid DNA) to deliver the expression vector to skin cells such that a transgenic protein is expressed may be evaluated (See, Example 3). Likewise, the ability of the nanoemulsion formulations of the present invention to deliver other biological agents into the skin (permeation rates) may be determined using labelled markers and permeation assays (See, Example 5). Furthermore, the ability of similar nanoemulsion formulations to deliver biological agents to the skin may be compared. Other assays may also employed to determine the ability of the nanoemulsion formulations of the present invention to deliver biological agents including, but not limited to, in vitro cell based assays and in vivo studies involving animal models or human subjects.

II. Biological Agents

In therapeutic and diagnostic embodiments of the present invention, the nanoemulsion formulations comprise biological agent(s). The nanoemulsion formulations of the present invention allow biological agents to penetrate natural barriers (e.g. skin), or increase the rate at which such penetration occurs, thus delivering or enhancing the therapeutic benefit of biological agents to a subject. These nanoemulsion formulations comprising biological agents may be employed to deliver these biological agents both parenterally and non-parenterally. The nanoemulsion formulations of the present invention may also be used in diagnostic, in vitro, and drug screening applications.

Examples of biological agents include, but are not limited to, nucleic acids such as DNA, cDNA, RNA (full length mRNA, ribozymes, antisense RNA, and decoys), oligodeoxynucleotides (phosphodiesters, phosphothioates, phosphoramidites, and all other chemical modifications), oligonucleotides, or linear and closed circular plasmid DNA; carbohydrates; proteins and peptides, including recombinant proteins such as for example cytokines (e.g. interleukens), trophic and growth or naturation factors (e.g. NGF, G-CSF, GM-CSF), enzymes, vaccines (e.g. HBsAG, gp120); vitamins, prostaglandins, drugs such as local anesthetics (e.g. procaine) antimalarial agents (e.g. chloroquine), anti-neoplastic agents (e.g. doxorubicin), antihistamines, biogenic amines (e.g. dopamine), antidepressants (e.g. desipramine), anticholinergics (e.g. atropine), antiarrhythimics (e.g. quinidine), antiemetics (e.g. chloroprimamine), antibiotics (e.g. penicillin) and analgesics (e.g. codeine and morphine) or small molecular weight drugs such as cisplatin that enhance transfection activity, or prolong the life of DNA in and outside of cells.

In some embodiments, where the biological agent is a antigenic protein or peptide, the nanoemulsion formulations of the present invention may be utilized as vaccines. In some embodiments, the presence of the oil component in the nanoemulsion formulation (vaccine) serves as an adjuvant.

In preferred embodiments, the biological agent are negatively or positively charged molecules, compounds or compositions. For example, negatively charged nucleic acids, negatively charged proteins and carbohydrates (including polysaccharides), and negatively charged drugs are mixed with nanoemulsion formulations of the present invention.

In some embodiments, the present invention provides a composition comprising a nanoemulsion formulation with a skin permeation rate of at least 0.447% per hour for a biological agent in the nanoemulsion formulation. In other embodiments, the nanoemulsion formulation has a skin permeation rate of at least 0.519% per hour for a biological agent in the nanoemulsion formulation. In a different embodiment, the nanoemulsion formulation has a skin permeation rate of at least 0.615% per hour for a biological agent in the nanoemulsion formulation. In preferred embodiments, the nanoemulsion has a skin permeation rate of at least 0.823% per hour for a biological agent in the nanoemulsion formulation. In particularly preferred embodiments, the nanoemulsion has a skin permeation rate of approximately 0.870% per hour for a biological agent in nanoemulsion formulation. In certain embodiments, the nanoemulsion formulation comprises a biological agent. In certain embodiments, the biological agent is a protein, carbohydrate, lipid, nucleic acid, or mixture thereof.

The present invention provides nanoemulsion formulations that are antimicrobial. The present invention contemplates that any antimicrobial may find use with the present invention. Any agent that can kill, inhibit, or otherwise attenuate the function of a microorganism may be used, as well as any agent contemplated to have such activities may be added to the nanoemulsion formulations of the present invention. Antimicrobial agents include, but are not limited to, natural and synthetic antibiotics, antibodies, inhibitory proteins, antisense nucleic acids, membrane disruptive agents and the like, used alone or in combination. Indeed, any type of antibiotic may be used including, but not limited to, anti-bacterial agents, anti-viral agents, anti-fungal agents, and the like.

In certain preferred embodiments the biological agents are nucleic acids. In this regard, the nanoemulsion formulations of the present invention deliver the nucleic acids to a subject in a diagnostic or therapeutic manner. The nucleic acids that may be delivered by the nanoemulsion formulations of the present invention include, but are not limited to: DNA, cDNA, RNA (full length mRNA, ribozymes, antisense RNA, and decoys), oligodeoxynucleotides (phosphodiesters, phosphothioates, phosphoramidites, and all other chemical modifications), oligonucleotide, linear and closed circular plasmid DNA, nucleic acids that encode a gene or gene fragment.

The nanoemulsions formulations of the present invention may comprise expression vectors (e.g. plasmids). In this regard, the nanoemulsion formulations may be used as delivery vehicles for gene therapy (e.g. expressing transgenes in the skin of a subject). In preferred embodiments, the genes to be introduced for gene therapy by the nanoemulsion formulations of the present invention generally fall into one of four categories. In the first are those genes that are intended to overcome a gene deficiency or defect in the subject (i.e., where the subject fails to produce active, endogenous protein at all or within normal levels, and the gene introduced in the plasmid is intended to make up this deficiency). Examples of this class of genes include genes encoding adenosine deaminase (ADA), for gene expression in stem cells or lymphocytes; genes encoding purine nucleoside phosphorylase deficiency, deficiency in prostaglandin G/H synthase, therapy of Lesch-Nyhan syndrome caused by a deficiency in hypoxanthine-guanine phosphoribosyltransferase, genes encoding a variety of circulating proteins, such as α₁-antitrypsin, clotting factors (e.g., Factor VIII, Factor IX) and globins (e.g., β-globin, hemoglobin), for the treatment of hemophilia, sickle-cell anemia and other blood-related diseases, and genes encoding hormones and other peptide regulators.

In the second class are polypeptides designed to treat any existing pathology, such as cancer, or a pathogenic condition such as viral infection. Examples include gene therapy to supply the p53 gene for cancer therapy, the gene for the CD4 peptide to inhibit HIV infection, the gene for the Pseudomonas peptide to inhibit binding of Pseudomonas to epithelial cells, and specific antibody genes to inhibit a targeted pathogen. The third class includes genes intended to produce an mRNA transcript that can act as an antisense molecule to inhibit an undesirable protein expression, such as overexpression of proteins specific for tumor growth, or expression of viral proteins (see below). The fourth class serves as a diagnostic or in drug screening assays. Diagnostic genes may be useful, for example, as marker genes to track expression, in test systems to determine transfection efficiency, in test systems to optimize expression, and in the introduction or removal of a gene. Also in the fourth class are gene used as part of drug screen, for example, introduction of a recombinase to excise a gene in a test animal flanked by site-specific recombination sites to create a gene knock out, followed by the introduction of compounds to test their ability to compensate for the knock out.

In some embodiments, the present invention provides a composition comprising a nanoemulsion formulation that permits an expression vector (e.g. plasmid) to express a recombinant peptide at a mean level of at least 57.0 pg.cm² in the cells of a subject. In other embodiments, the nanoemulsion formulation permits an expression vector to express a recombinant peptide at a mean level of at least 100.0 pg/cm² in the cells of a subject. In other embodiments, the nanoemulsion formulations permits an expression vector to express a recombinant peptide at a mean level of at least 285.0 pg/cm², or at a mean level of at least 376.0 pg/cm². In other embodiments, the cells of the subject are skin cells. In certain embodiments, the recombinant peptide expressed by the expression vector is human interferon-α2.

In some embodiments, the present invention provides a composition comprising a nanoemulsion formulation that permits an expression vector (e.g. plasmid) to express RNA transcripts at a level of at least 5.0×10⁴ transcripts/cm² in cells of a subject. In other embodiments, the nanoemulsion formulation permits an expression vector to express RNA transcripts at a level of at least 5.0×10⁵ transcripts/cm² in the cells of a subject. In other embodiments, the cells of the subject are skin cells. In some embodiments, the RNA transcripts expressed by the expression vector are antisense RNA molecules.

In further embodiments, the expression vector (e.g. plasmid) expresses a recombinant peptide (e.g. protein, peptide, glycoprotein) when it has been administered to a subject in the nanoemulsion formulations of the present invention such that the recombinant peptide serves as a vaccine. In this regard, the expressed recombinant peptide may be an antigen capable of triggering in said subject an immune reaction with respect to a pathogenic virus (e.g. pathogenic viruses, the Aujesky virus, an HIV virus such as HIV-I or HIV-II, an FIV virus or a flu virus of the influenza type) or to a pathogenic microorganism (e.g. bacterium, a yeast, a fungus, a mycoplasma or a unicellular parasite). This immune reaction may be of the humoral or cellular type, and may consist, in particular, of a synthesis of antibody enabling a protection with respect to such a virus or such a microorganism to be conferred on the host body. Usually, the coding sequence in the expression vector originates from such a pathogenic virus or microorganism.

The nanoemulsion formulations of the present invention may comprise oligonucleotides for use in antisense modulation of the function of DNA or messenger RNA (mRNA) encoding a protein the modulation of which is desired, and ultimately to regulate the amount of such a protein. Hybridization of an antisense oligonucleotide with its mRNA target interferes with the normal role of mRNA and causes a modulation of its function in cells. The functions of mRNA to be interfered with include all vital functions such as translocation of the RNA to the site for protein translation, actual translation of protein from the RNA, splicing of the RNA to yield one or more mRNA species, turnover or degradation of the mRNA and possibly independent catalytic activity which may be engaged in by the RNA. The overall effect of such interference with mRNA function is modulation of the expression of a protein. In the context of the present invention, “modulation” means either an increase (stimulation) or a decrease (inhibition) in the expression of a gene. In the context of the present invention, inhibition is the preferred form of modulation of gene expression and mRNA is a preferred target.

Antisense compounds are commonly used as research reagents diagnostic aids, and therapeutic agents. For example, antisense oligonucleotides, which are able to inhibit gene expression with specificity, can be used to elucidate the function of particular genes. Antisense compounds are also used, for example, to distinguish between functions of various members of a biological pathway.

While antisense oligonucleotides are a preferred form of antisense compound, the present invention comprehends other oligomeric antisense compounds, including but not limited to oligonucleotide mimetics. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

The nanoemulsion formulations of the present invention may also comprise nucleic acid molecules selectively screened to bind a selected target. For example, screening may conducted using the technique known as SELEX. The basic SELEX procedure is described in U.S. Pat. Nos. 5,475,096 and 5,270,163 (herein incorporated by reference in their entireties). The SELEX procedure allows identification of a nucleic acid molecules with unique sequences, each of which has the property of binding specifically to a desired target compound or molecule.

The nanoemulsion formulations of the present invention may further comprise other supplementary biological agents such as pharmaceutically acceptable carriers, or diluents. Example of pharmaceutically acceptable carriers include, but are not limited to, a liquid, cream, foam, lotion, or gel, and may additionally comprise organic solvents, emulsifiers, gelling agents, moisturizers, stabilizers, wetting agents, preservatives, time release agents, and minor amounts of humectants, sequestering agents, dyes, perfumes, and other components commonly employed in pharmaceutical compositions.

In some embodiments, the nanoemulsion formulations of the present invention further comprise dendrimer molecules. Dendrimeric polymers have been described extensively (See, Tomalia, Advanced Materials 6:529 [1994]; Angew, Chem. Int. Ed. Engl., 29:138 [1990]; incorporated herein by reference in their entireties). Dendrimer polymers are synthesized as defined spherical structures. Molecular weight and the number of terminal groups increase exponentially as a function of generation (the number of layers) of the polymer. Different types of dendrimers can be synthesized based on the core structure that initiates the polymerization process. In some embodiments, the dendrimer molecules are conjugated to one or more biological agents. In other embodiments, the dendrimer molecules are employed to increase the ability of the nanoemulsion formulations to penetrate biological membranes (e.g. skin).

The nanoemulsion formulations of the present invention are capable of delivering a biological agent to a subject. In this regard, the present invention contemplates treatment for any type of disease or condition that may be treated by a biological agent. In preferred embodiments, diseases that are amenable to local treatment (e.g. the skin, and mucous membranes) are treated by the nanoemulsion formulations of the present invention. Examples of such diseases, include, but are not limited to; cancer, autoimmune diseases, hair follicle diseases, subacute cutaneous lupus erythematosus, and discoid lupus erythematosus. Other examples include, but are not limited to, the treatment of Kaposi's sarcoma and systemic lupus with nanoemulsions containing (or causing the expression of) human interferon-α2.

The nanoemulsions of the present invention may also be delivered to cells and subjects for diagnostic applications. The nanoemulsions comprising biological agents may be used in vitro to monitor the effect of certain biological agents on cells or to monitor cellular processes. In one embodiment, cells are cultured in vitro with a nanoemulsion formulation comprising a biological agent, and the effect of the biological agent is determined. The nanoemulsion formulations comprising biological agents may be administered to a subject to monitor cellular processes in the subject or to monitor cellular events. In some embodiments, the biological agent is a dye or other detectable marker.

III. Modes of Administration

The nanoemulsion formulations of the present invention may administered in any acceptable manner. In some embodiments, the nanoemulsion formulations of the present invention are delivered to a subject by parenteral administration. Parenteral administration includes, but is not limited to, administration intravenously, intra-muscularly, subcutaneously, intradermally, intraperitoneally, intrapleurally, or intrathecally.

In certain preferred embodiments, the nanoemulsion formulations of the present invention are delivered to a subject by non-parenteral routes of administration. Non-parenteral administration refers to the administration, directly or otherwise, of the nanoemulsion formulations of the present invention via a non-invasive procedure which typically does not entail the use of a syringe and needle. Non-parenteral administration includes, but is not limited to, the contacting, directly or otherwise, to all or a portion of the alimentary canal, skin, eyes, pulmonary tract, urethra or vagina of an animal. Specific examples of non-parenteral administration, include, but are not limited to, buccal, sublingual, endoscopic, oral, rectal, transdermal, nasal, intratracheal, pulmonary, urethral, vaginal, ocular, and topical.

The alimentary canal is the tubular passage in animal that functions in the digestion and absorption of food and the elimination of food residue, which runs from the mouth to the anus, and any and all of its portions or segments (e.g. the oral cavity, the esophagus, the stomach, the small and large intestines and the colon, as well as compound portions thereof like the gastro-intestinal tract. Therefore, delivery to the alimentary canal encompasses several routes of administration including, but not limited to, oral, rectal, endoscopic and sublingual/buccal administration.

In some embodiments, the non-parenteral administration of the nanoemulsion formulations of the present invention may include iontophoresis (the transfer of ionic solutes through biological membranes under the influence of an electric field), phonophoresis or sonophoresis (use of ultrasound to enhance the absorption of various therapeutic agents across biological membrane, notably the skin and cornea). These techniques may be used to enhance the transport of the nanoemulsion formulations of the present invention such that biological agents in the nanoemulsion formulations are able to have a therapeutic effect.

Delivery of the nanoemulsion formulations of the present invention via the oral mucosa, as in the case of buccal and sublingual administration, has several desirable features, including, in many instances, a more rapid rise in plasma concentrations of the biological agents, than via oral delivery. Furthermore, because venous drainage from the mouth is to the superior vena cava, this route also bypasses rapid first-pass metabolism by the liver.

Endoscopy may be used for delivery of the nanoemulsion formulations of the present invention directly to an interior portion of the alimentary tract. For example, endoscopic retrograde cystopancreatography (ERCP) takes advantage of extended gastroscopy and permits selective access to the biliary tract and the pancreatic duct. The nanoemulsion formulations of the present invention can be delivered directly into portions of the alimentary canal (e.g. duodenum or gastric submucosa) via endoscopic means. Gastric lavage devices and percutaneous endoscopic feeding devices (Pennington et al., Ailment Pharmacol. Ther., 1995) can also be used for direct alimentary canal delivery of nanoemulsion formulations.

The nanoemulsion formulations of the present invention may be administered by the lower enteral route (e.g,. through the anus into the rectum or lower intestine). Rectal suppositories, retention enemas or rectal catheters can be used for this purposed and may be preferred if this is the site of disease or patient compliance might otherwise be hard to achieve (e.g. pediatric, geriatric, or unconscience patients).

In particularly preferred embodiments of the present invention, the nanoemulsion formulations are delivered topically (locally) to a subject. Topical application of the nanoemulsion formulations primarily produces local effects. Examples of topical application include, but are not limited to, topical application to mucous membranes, skin, eyes, or to organ surfaces (either ex vivo transplant organs or in vivo organs). A preferred topical route of administration is through the skin.

Nanoemulsion formulations applied to mucous membranes produce primarily local effects. This route of administration includes application of the nanoemulsion formulations to mucous membranes of the conjunctiva, nasopharynx, oropharynx, vagina, colon, urethra, and urinary bladder. Ocular delivery of the nanoemulsion formulations of the present invention is useful for the local treatment of eye infections or abnormalities. The nanoemulsion formulation may be administered via instillation and absorption occurs through the cornea. Comeal infection or trauma may thus result in more rapid absorption.

A preferred mode of local administration of the nanoemulsion formulations of the present invention is through the skin of a subject (i.e. topical application of the nanoemulsion formulations to a subject's skin). Topical delivery of the nanoemulsion formulations of the present invention has the advantage of directing the biological agents in the nanoemulsion formulations to the confined site of disease (e.g. clinically active skin lesions). Topical application to the skin also prevents any adverse toxic side effects that may be caused by systemic application of the nanoemulsion formulations of the present invention. Topical application of the nanoemulsion formulations of the present invention may be, for example, in the form of a transdermal patch, impregnated into absorptive materials, such as sutures, bandages, and gauze, or coated onto the surface of solid phase materials. In preferred embodiments, the administration of the nanoemulsion formulations to a subjects skin in non-irritating to the skin.

In preferred embodiments, the nanoemulsion formulations are administered to the skin via a transdermal patch. While not limited to any mechanism, it is believed that topical delivery to the skin via a transdermal patch provides a continuous supply of the nanoemulsion formulations, maintaining a steady supply of biological agents, to achieve the desired biological effect. Transdermal delivery may be more convenient than other modes of delivery (especially for children), and could increase patient compliance. One example of a transdermal patch for delivering biological agents in liposome formulations is found in U.S. Pat. No. 5,718,914, herein incorporated by reference. Other transdermal patches are known, and are contemplated as modes for delivering the nanoemulsion formulations of the present invention.

IV.) Kits

In some embodiments of the present invention, the components of the nanoemulsion formulations and desired biological agents are separated into individual formulations (e.g. individual vials) for later mixing during use, as may be desired for a particular application. Such components may advantageously be placed in kits for diagnostic or therapeutic use. In some embodiment, such kits contain all the essential materials and reagents required for the delivery of biological agents via the nanoemulsion formulations of the present invention to the site of their intended action. In some embodiments, the kits comprise fully assembled formulations.

The kits of the present invention may also include a means for containing the vials in close confinement for commercial sale (e.g., injection or blow-molded plastic containers into which the desired vials are retained). Irrespective of the number or type of containers, the kits of the invention also may comprise, or be packaged with, an instrument for assisting with the administration or placement of the nanoemulsion formulation on or in a subject. Examples of such instruments include, but are not limited to, inhalers, syringes, pipettes, forceps, measured spoons, eyedroppers, swabs, patches, or any such medically approved delivery vehicle.

Experimental

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); ° C. (degrees Centigrade); and Sigma (Sigma Chemical Co., St. Louis, Mo.).

### EXAMPLE 1

### Preparation of Nanoemulsions Formulations Containing Plasmids

This examples describes the preparation of certain nanoemulsion formulations containing plasmids. In particular, this example describes the preparation of nanoemulsion formulations with expression plasmids encoding either chloramphenicol acetyltransferase (CAT) or human interferon-α2 (huINFα2).

The CAT expression plasmid employed was pCF1CAT (Raczka, et al., Gene Ther., 5:1333 [1998]), and the huINFα2 expression plasmid employed was pNGVL3huINFα2, which consists of the cDNA for human interferon-α2 cloned into the polylinker of pNGVL3 (Sant et al., Hum. Gene Ther., 9:2735 [1998]). These expression plasmids were amplified in E. coli bacteria and then isolated by double cesium chloride gradients. This was followed by dialysis against sterile Tris-EDTA buffer (to reduce endotoxin contamination) thus forming aqueous plasmid solutions.

Each nanoemulsion formulation was formed by combining 18 parts by volume polyoxyethylene sorbitan monooleate (Tween 80, Sigma), 30 parts by volume sorbitan monooleate (Span 80, Sigma), and 46 parts by volume olive oil (Sigma) (in order to form 5:3 Span 80:Tween 80 nanoemulsions). These surfactant—oil mixtures were then warmed to 50° C. and sterile filtered with a 0.22 μm filter into sterile microfuge tubes. Next, to each surfactant—oil mixture 6 parts by volume of aqueous plasmid solution (CAT or huINFα2) was added and mixed gently to yield a clear nanoemulsion with CAT plasmids or huINFα2 plasmids. The concentration of pCF1CAT and pNGVL3huINFα2 in the respective nanoemulsion formulations was held constant at 0.2 mg/ml total volume. Blank nanoemulsion formulations containing no plasmid DNA were also prepared, along with aqueous plasmid DNA solutions that served as controls.

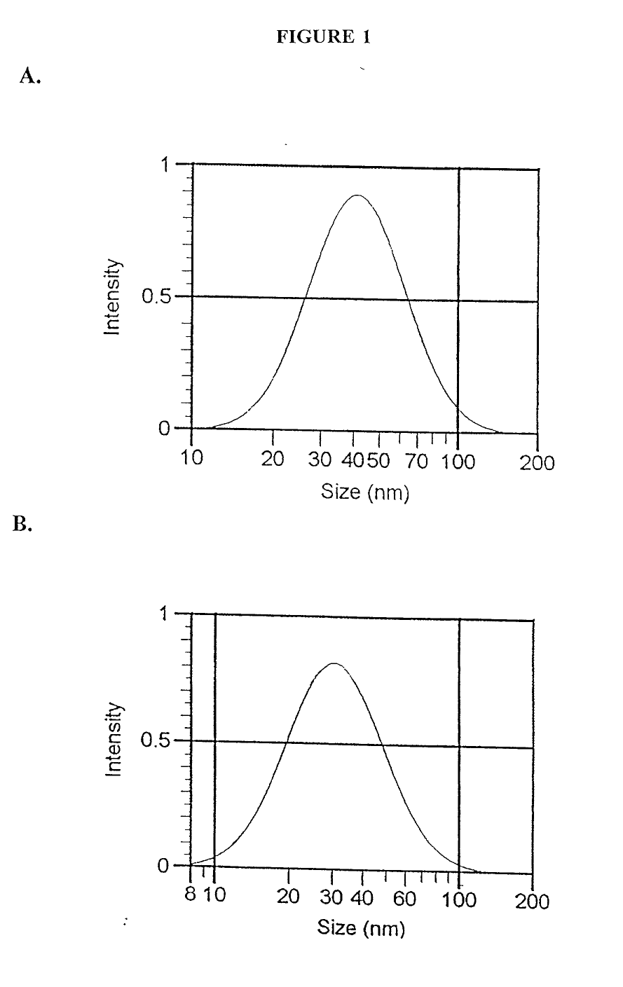

The CAT plasmid nanoemulsion formulations and the blank nanoemulsion formulations were characterized by particle size distribution analysis using a Beckman Coulter N4 Plus Submicron Particle Size Analyzer utilizing photon correlation spectroscopy. The particle size analysis (See, FIG. 1) indicated that the nanoemulsion formulation without DNA had a mean particle size of 42.3+/−14.6 nm (FIG. 1A), while the nanoemulsion formulation with DNA (CAT plasmid) (FIG. 1B) had a mean particle size of 32.1+/−20.2 nm. These results suggest that the plasmid DNA in the nanoemulsion formulations is in a condensed state, as plasmid DNA in an aqueous solution normally has a particle size on the order of 50-100 nm. In addition, the electroconductivity of the CAT plasmid nanoemulsion formulation and the blank nanoemulsion formulation was evaluated using a Beckman Coutler Delsa. Both of these nanoemulsion formulations exhibited no electroconductivity at a field potential of>300 V.

### Example 2

### Preparation of Plasmid-Liposome Formulations

This examples describes the preparation of plasmid-liposome formulations. The liposome formulations were created by combining glycerly dilaurate (GDL), polyoxyethylene-10-stearyl ether (POE-10), cholesterol (CH), and 1,2-Dioleyl-3-trimethylammonium propane (DOTAP) at a weight percent ration of 50:15:23:12 respectively. GDL, POE-10, and CH were all purchased from IGI, Inc. (Little Falls, N.J.), and DOTOP was purchased from Avanti Polar Lipids, Inc. (Alabaster, Ala.). The appropriate amounts of these lipids were mixed and melted at 70° C. in a sterile polystyrene centrifuge tube. The lipid melt was then filtered through a 0.22 μm filter (Nuclepore) and the filtrate was reheated in a water-bath at 70° C. prior to being drawn into a sterile syringe. A second syringe containing sterile, autoclaved, double-distilled water was preheated to 65° C. and connected via a 3-way sterile stopcock to the lipid phase syringe. The aqueous phase was then slowly injected into the lipid phase syringe. The mixture was rapidly passed back and forth between the two syringes while being cooled under cold tap water until the mixture was at room temperature.

The resulting liposomal suspension (with a total lipid concentration of 100 mg/ml) was examined using a Nikon Diaphot light microscope to assure integrity and quality. The liposomal suspension was then sonicated for 20 minutes at room temperature, and the particle size of the sonicated liposomes was determined using a NiComp 370 Particle Sizer. The desired size of the sonicated liposomes was 100-140 nm, which was found to be the size of the sonicated liposomes in this example with a narrow distribution (polydispersity index between 0.2-0.3). Sonicated liposomes not falling into this range were further sonicated until the size limitation was met. Appropriate amounts of an aqueous plasmid pCF1CAT solution (See, Example 1) were then added to the sonicated NC liposomes by inversion mixing to obtain plasmid-liposome formulations containing DOTAP:DNA weight ratios of 2:1, 4:1, 6:1, 8:1, and 10:1.

### Example 3

### In vivo Transfection of Mice with Plasmid Nanoemulsion Formulations

This example describes the in vivo transfection of mice with plasmid nanoemulsion formulations. In particular, this example describes the topical administration of the CAT plasmid and huINFα2 plasmid nanoemulsion formulations (described above in Example 1) to mice. This example also describes processing the murine skin after administration of the plasmid nanoemulsion formulations, as well as various assays performed on the murine skin samples (e.g. ELISA, In Situ PCR, RT-PCR, and histological examination).

A. In vivo Transfection of Mice

In vivo transfection was performed through single dose or multiple dose topical administration of plasmid nanoemulsion formulations (CAT or huINFα2) to mice. For single dose experiments, male hairless mice (Skh-hr-1, 60 days old, Charles River Breeding Laboratories, Wilmington, Del.) were used, and in the multiple dosing experiments male hairy mice (C57 BL/6J, 60 days old, Jackson Laboratory) were used. A glass donor cap with an area of 1.1 cm² was affixed to the skin on the dorsal side of each mouse by application of cyanoacrylate adhesive to the perimeter of the cap. In the case of the C57 BL/6J mice, hair was removed with electric trimmers prior to adhesion of donor caps to the skin.

In the single dose experiments 20, 50, or 200 μl of the plasmid nanoemulsion formulation was placed on the skin circumscribed within the donor cap and spread evenly using a pipette tip to achieve complete surface coverage. These volumes correspond to three different total doses of plasmid DNA (3, 10, and 30 μg respectively) that were used in order to examine does response effects. In the multiple dosing experiments 50 μl of the plasmid nanoemulsion was applied to the murine skin within the donor caps. In every case, after the application of the plasmid nanoemulsion, the donor cap was then occluded with Parafilm and the entire area was tightly wrapped with COBAN self-adherent wrap (3M Health Care, St. Paul, Minn.). In the multiple dosing experiments, animals were retreated every 24 hours and any remaining nanoemulsion formulation was removed with KIMWIPES prior to the application of a fresh 50 μl of the plasmid nanoemulsion formulation. This procedure was carried out for four consecutive days. Six to ten animals per group were examined in the single dose experiments and four animals per group were examined in the multiple dosing experiments.

B. Processing of Murine Skin

In order to determine the effects of the nanoemulsion formulations the skin of the tested mice, the mice were sacrificed with a lethal dose injection of sodium pentobarbital, the occlusive dressing and donor cap were removed, and the skin was excised using sharp dissection. Subcutaneous fat was removed the skin placed on a wooden board, epidermis up and secured with push pins. Multiple 4 mm diameter punch biopsies (Miltex, Inc.) were then obtained from the area of skin exposed to treatment. Seven to ten biopsies were typically collected from the treated area and placed as groups of three into sterile Eppendorf tubes. One group was used for quantitative RT-PCR, and a second group was used for ELISA. Both groups of samples were snap frozen in dry ice-ethanol slurry and stored at −70° C. until the extraction procedures were undertaken. A third group was immersion fixed in 10% buffered formalin and processed for paraffin sectioning.

C. RT-PCR Assay for huINFα2 Nanoemulsion Formulations

Isolation of total RNA was accomplished by homogenization of snap frozen skin biopsies in Trizol reagent (GibcoBRL Life Technologies, Bethesda, Md.) followed by precipitation in isopropanol. Poly A RNA was then isolated using oligo (dT)-cellulose columns (Qiagen) and immediately converted to single stranded cDNA using oligo (dT) (12-18) and M-MLV reverse transcriptase (GibcoBRL Life Technologies). Samples were ethanol precipitated, re-suspended in Tris-EDTA buffer, quantified by spectrophotometry and frozen at -80° C. until tested in RT-PCR.

The skin samples were analyzed using a real time quantitative RT-PCR assay (TaqMan, Applied Biosystems, Brachburg, N.J.) specific for human interferon-α mRNA. The quantitative RT-PCR assay used a forward primer (5′-TTTAGTGAACCGCACCGTCGTCG-3′, SEQ ID NO:1) that spanned the splice donor and splice acceptor of the CMV intron within pNGVL3, including sequences from 675-1066 of pNGVL3huINFα2. This was done to reduce the possibility of false positive signals arising from inadvertent priming with residual plasmid DNA present in the recovered skin samples. The reverse transcription primer (5′-GAGATTCTCCTCATCTGTGCCAGG-3′, SEQ ID NO:2) was specific for sequences identified in the human interferon-α2 cDNA and had no significant overlap with cDNA sequences for any reported murine interferon-α (NCBI BLAST search). The sequence of the indicator primer was 5′ FAM-CCCACAGCCTGGGTAGCAGGAGGACC-TAMRA 3′, SEQ ID NO:3) (MegaBases, Inc., Evanston, Ill.). One μg of single stranded cDNA from each sample was used as a template. Known quantities of purified pNGVL3huINFα2 were used to generate standard curves with linearity from 10³ to 10⁸ sequences per μl.

The RT-PCR assay described above was employed to detect the presence of the expression plasmid transcripts in the skin of both hairless (Skh-hr-l) and normal mice (C57 BL/6J) following the application (single or multiple dose) of the huINFα2 nanoemulsion formulation. The results of these assay were expressed as huINFα2 transcripts/cm². The normal mice had a mean value of 5.0×10⁵ huINFα2 transcripts/cm², while the hairless mice had a mean value of 2.0×10⁴ huINFα2 transcripts/cm² (a difference that was not statistically significant).

D. ELISA Assay for CAT and huINFα2 Nanoemulsion Formulations Isolation of total protein was accomplished by adding 100 μl of lysis buffer (Boehringer Mannheim, Indianapolis, Ind.) to each tube containing the skin tissue samples and vortexing for a few seconds. The tubes containing skin tissue were kept on ice. A probe sonicator (Micro Ultrasonic Cell Disruptor, Kontes, Inc.) was used to disrupt the skin in each tube under the following conditions: 40 W and Ouput 60 (range 0 to 100). The samples were sonicated two times and each sonication consisted of 7 pulses. The interval between the two sonications was about 10 minutes. The samples were then centrifuged at 15,000 rpm and 4° C. for 20 minutes. The supernatant from each tube for the same animal sample was then combined for ELISA determinations. To extract the CAT protein from skin tissue completely, the skin samples were sonicated again for a total of four times using the conditions described above after the above process was completed. The supernatants from the additional extractions were analyzed separately.

The quantitative determination of chloramphenicol acetyltransferase (CAT) and huINFα2 expression was carried out using a colorimetric enzyme immunoassay. CAT and huINFα2 ELISA kits (CAT, Boehringer Mannheim, Indianapolis, Ind., huINFα2, PBL Biomedical Inc., New Brunswick, N.J.) were used according to the manufacturer's instructions. Tissue homogenates were assayed in triplicate and the optical density of each sample was determined using a spectrophotometer set to 450 nm. A standard curve was also prepared using the homogenate buffer as the diluent from 0 pg/ml to 1000 pg/ml. The detection limit of the CAT ELISA was 10 pg/ml, and that of the huINFα2 ELISA was 12.5 pg/ml. The test samples were compared to the standard curve to determine the concentrations of transgenic protein. Standard curves for total protein assay were obtained using standard BSA solutions. BCA (Bicinchonomic acid protein assay reagent, Pierce, Rockford, Ill.) was used for the measurement of total protein in the supernatant of each sample. Additional controls consisted of untreated skin spikes with known amounts of recombinant human interferon-α2, and in all cases the spikes were recovered by the ELISA at predicted levels.

The ELISA assays describe above were employed for five different transgene expression determinations: i.) emulsion dose response studies; ii) time course studies; iii.) liposome dose response studies; iv.) normal versus hairless mouse skin studies; and v.) emulsion multiple dose response studies. These results were expressed as means of pg transgenic protein/cm² of treated skin and/or pg transgenic protein/mg total protein±standard deviations. Differences in mean values and standard deviations were analyzed using Student's T test to determine levels of statistical significance.

i.) Emulsion Dose Response Studies

The results of dose-response studies assayed at 24 hours following in vivo topical application of various CAT plasmid nanoemulsion formulations to hairless mouse skin at DNA doses of 3, 10, and 30 μg, are shown in Table 1. Values are expressed as means±standard deviations, and samples that exhibited mean levels of CAT that were below the limits of detection of the ELISA are expressed as <10.

TABLE 2
Dose Response 24 Hours Following Topical Application of Nanoemulsions

| Dose of pCF1CAT | 3 μg | 3 μg | 10 μg | 10 μg | 30 μg | 30 μg |
|---|---|---|---|---|---|---|
| Levels of CAT Protein | pg/cm² | pg/mg* | pg/cm² | pg/mg* | pg/cm² | pg/mg* |
| Topical Formulations | | | | | | |
| CAT Nanoemulsion | <10 | <10 | 376 ± 49 | 285 ± 201 | 290 ± 144 | 140 ± 87 |
| Aqueous DNA | <10 | <10 | 31 ± 12 | 53 ± 46 | <10 | <10 |
| Empty Nanoemulsion | <10 | <10 | <10 | <10 | <10 | <10 |

*total protein

No significant transgene expression was observed at a total DNA dose of 3 μg regardless of the nanoemulsion formulation used. At a total DNA dose of 10 1 μg insignificant levels of transgene expression were observed in both the aqueous DNA and nanoemulsion formulation DNA groups. However, the levels of CAT expression achieved using the nanoemulsion DNA formulation were significantly higher (an approximately 1 log increase) than those observed using an equivalent dose of aqueous DNA (p<0.001). It was also observed that increasing the total DNA dose to 30 μg (using either aqueous DNA or a nanoemulsion formulation), did not enhance transgene expression. In fact, at a DNA dose of 30 μg an aqueous formulation was unable to mediate detectable levels of transgene expression. These results suggest that topical transfection, regardless of the drug delivery system used is subject to both threshold and saturation effects associated with the total dose of DNA applied.

ii.) Time Course Studies

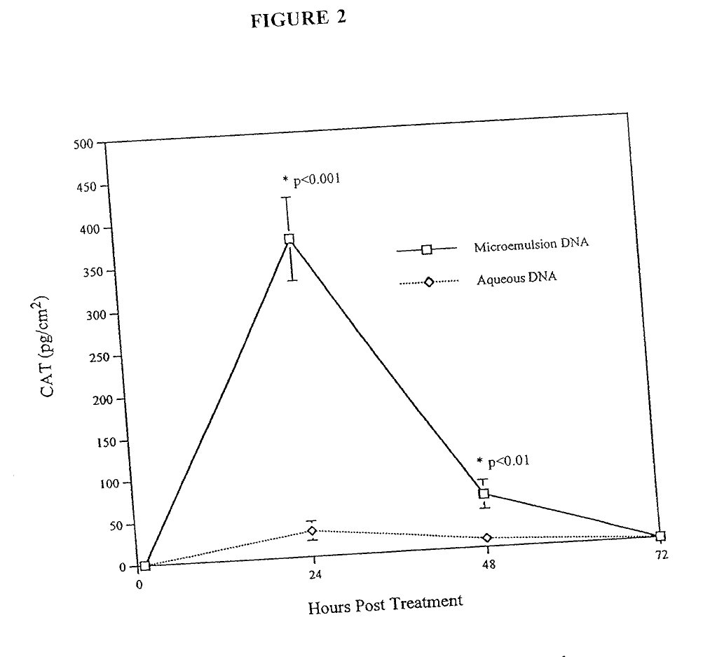

The relative time course of transgene expression following application of a single dose of aqueous DNA or nanoemulsions DNA is illustrated in FIG. 2. Consistent with previous data using both liposomal systems and aqueous DNA (Niemiec, et al., J. Pharm Sci., 86:(6)701-708 [1997]; Yu et al., J. Invest. Dermatol, 112:370-375 (1999); and Fan et al., Nat. Biotechnology, 17:870-872, [1999]), the observed levels of transgene expression were highest 24 hours following application of the topical formulations and had returned to baseline by 72 hours. The levels of transgene expression observed using the DNA nanoemulsion formulations were significantly greater than those observed using aqueous DNA at both 24 and 48 hours (p<0.001 and p<0.01 respectively).

iii.) Liposome Dose Response Studies

Five different liposomal formulations were administered to, with DOTAP:CAT plasmid (w/w) ratios of 2:1, 4:1, 6:1, 8:1, and 10:1, and the dose response of the skin samples was measured by ELISA. It was determined that none of the liposomal formulations tested were able to mediate levels of CAT transgene expression in skin that were above the limits of detection for the ELISA employed (which was 10 pg/ml).

iv.) Normal Versus Hairless Mouse Skin Studies

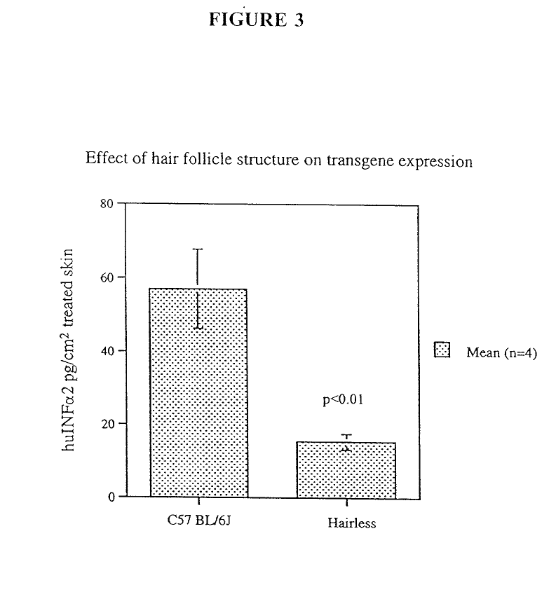

Comparisons were made between groups of normal mice (C57 BL/6J) and hairless mice (Skh-hr-1) (n=4) that were treated with a single dose of huINFα2 nanoemulsion formulations containing 10 μg of pNGVL3huINFα2 plasmid DNA in a total volume of 100 μl. The treated skin was harvested 24 hours following dosing and analyzed using ELISA for human interferon-α2 protein. As shown in FIG. 3 the normal C57 BL/6J mice expressed a mean value of 57.0±2.2 pg huINFα2/cm² of treated skin, while the hairless mice expressed a mean value of 15.4±2.2. pg huINFα2/cm² of treated skin (p=0.01). These experiments suggest that the level of transgene expression that can be achieved in skin with normal follicular structure is higher than that observed in the abnormal follicles in the hairless mice skin.

v.) Emulsion Multiple Dose Response Studies

In normal C57 BL/6J mice treated with four daily doses of the huINFα2 nanoemulsions, the skin contained human interferonα2 at an average level of 5.2±0.98 pg of interferon-α2 protein per mg of total protein, or approximately 100 pg interferon-α2/cm² of treated skin. Control mice treated with empty nanoemulsions had mean values below the limits of detection for the ELISA (p<0.001). The total amount of transgenic protein present in the treated skin also appeared to increase over the course of multiple topical applications (approximately 50 pg/cm², 24 hours following a single application, versus 100 pg/cm², 24 hours following four daily applications). This indicates that multiple topical applications of the plasmid nanoemulsion formulations allows for continuous transgene expression during the entire treatment period.

E. In Situ PCR Results for huINFα2 Nanoemulsions

In situ DNA PCR analysis of treated skin was conducted to identify intracellular plasmid DNA (pNGVL3huINFα2) 24 hours following topical in vivo application plasmid nanoemulsions to normal mouse skin. Direct in situ PCR was performed according to previously published methodology (Nuovo G. J., “PCR in situ Hybridization”, Raven Press, New York, pp. 54-246 (1994); and Foreman et al., J. Clin. Invest., 99:2971-2978 (1997)).

Oligonucleotide primers were designed so that amplified fragments would span intronic sequences within pNGVL3huINFα2 plasmid in order to prevent inadvertent amplification of expression plasmid transcripts. The sequence of the forward primer was 5′-TCCATGGGTCTTTTCAGCAGT-3′ (SEQ ID NO:4) and the sequence of the reverse primer was 5′-ATTCTCCTCATCTGTGCCAGG-3′ (SEQ ID NO:5). The length of the expected PCR fragment specific for the pNGVL3huINFα2 plasmid was 100 bp. Controls included sections assayed in the absence of oligonucleotide primers. PCR amplifications were performed using a GeneAmp PCR 1000 System (Perkin-Elmer Cetus Instruments) as follows: 92° C. for 1 minute, 65° C. for 1 minute, and 72° C. for 1 minute for a total of 40 cycles. Control reactions used for each sample included reaction mixtures without Taq polymerase, and/or primers, skin treated with aqueous DNA, skin treated with blank nanoemulsions as well as tissue samples from untreated mouse skin.

Anti digoxigenin-alkaline phosphatase-conjugated Fab fragments (1:2,000 dilution Boehringer Mannheim) were used for binding to in situ amplified PCR fragments and NBT/BCIP was used as a calorimetric detection reagent. Sections were lightly counterstained with dilute hematoxylin and examined using light photomicroscopy (Nikon Diaphot). Evidence of plasmid DNA (dark pigment) was identified within numerous hair follicles with extension of positive signals onto the perifollicular surface keratinocytes. No pigment was identified in areas of the sections that were non-follicular or in the remainder of the dermis. Skin sections from a variety of controls showed no evidence of specific expression plasmid DNA amplification. Additional controls included sections from all animals assayed in the absence of oligonucleotide primers (See, Nuovo G. J., “PCR in situ Hybridization”, Raven Press, New York, pp. 54-246 (1994); and Foreman et al., J. Clin. Invest., 99:2971-2978 (1997)). None of these sections exhibited any evidence of intracellular pigment deposition. These observations strongly suggest that the delivery of plasmids with nanoemulsion formulations into skin occurs predominantly via a follicular pathway. In addition, the distribution of pigment suggests that the plasmid DNA identified was present exclusively in an intracellular location.

F. Histology of Skin Samples

Representative paraffin sections (10 μm) were obtained from treated (four daily doses of aqueous huINFα2, blank nanoemulsion formulations, or huINFα2 nanoemulsion formulations) and untreated skin (sham), stained with hematoxylin/eosin and examined in blinded fashion by a veterinary pathologist for evidence of treatment specific irritation or inflammation. In particular, using a quantitative pathologic assessment, each section was evaluated for epidermal thickness, hyperkeratosis, serocellular crust, epidermal ulceration, and epidermal inflammatory infiltrates. In addition, the dermis and panniculus fat were examiner for the presence of cellular infiltrates, hemorrhages and congestion. No histologic differences were appreciated between sham treated animals and those treated with aqueous huINFα2, blank nanoemulsion formulations, or nanoemulsion formulations containing pNGVL3huINFα2 DNA.

### Example 4

### Nanoemulsion Phase Diagram Construction

This example describes the construction of various phase diagrams involving oil, surfactant mixtures, and water. In particular, phase diagrams were constructed by titrating a series of olive oil/surfactant mixtures (Span 80 and Tween 8) with water (double-distilled and de-ionized using a Millipore Milli-Q Water System, Millipore Corp., Bedford, Mass.) at ambient temperature to form various nanoemulsions.

Three different surfactant mixtures were made, including 1:1, 2:1, and 3:1 by volume of Span 80 to Tween 80. Typically, the surfactants were mixed at the desired ratio and allowed to equilibrate overnight. 2.5 ml of the surfactant mixture was placed in a 20 ml scintillation vial with a positive displacement pipet (Gilman). Distilled water was then added to the surfactant mixture within the vial in small aliquots of 25 μl. Following addition of the aliquot of water, the vial was capped and vortexed for 2 minutes to accelerate equilibration. Following vortexing, the mixture was visually examined for clarity. Titrations were carried out until the mixture became hazy or turbid to establish the region of clear isotropic mixtures along the water-surfactant axis in the pseudo-ternary diagram.

Next, small aliquots of olive oil were added to the surfactant-water to establish isotropic regions along the axis from the surfactant-water baseline towards the oil apex. If mixtures appeared hazy, small aliquots of the surfactant mixture was added until it became clear. This process was continued to determine the entire domain of clarity from an oil-poor isotropic phase to an oil-rich isotropic one. No attempt was made to distinguish between micelles, swollen micelles, oil-in-water nanoemulsions, water-in-oil nanoemulsions, bicontinous nanoemulsions, or liquid crystalline phases.

The desired system was placed in an eppendorf tube and centrifuged at 5000 rpm for 15 minutes at room temperature to determine its stability as an isotropic single phase system. The particle size of the clear formulations was also determined using a Nicomp 370 Submicron Particle Sizer (HIAC-Royco). A water soluble dye was used to qualitatively determine whether the isotropic systems were water-external or oil-external systems. Dilution tests with water or olive oil were also carried out to further qualitatively characterize the nature of the isotropic mixtures.

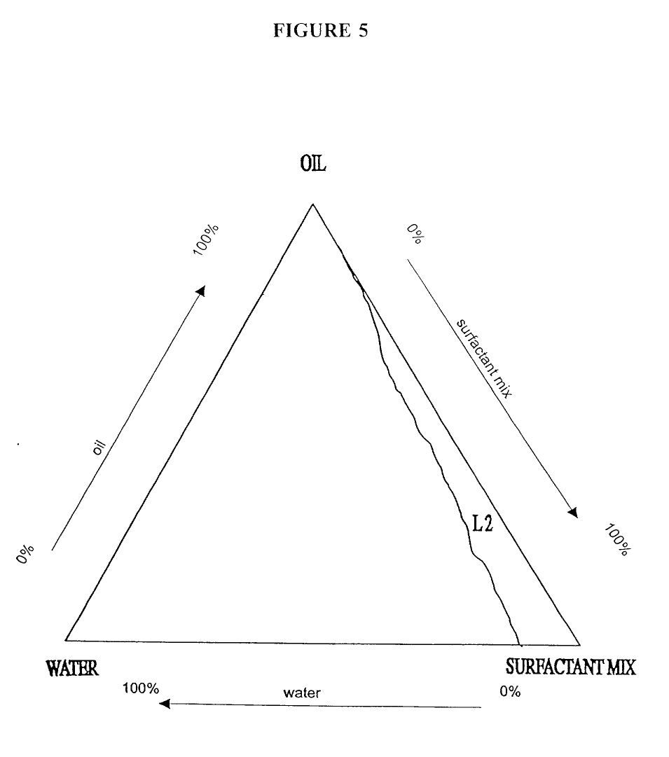

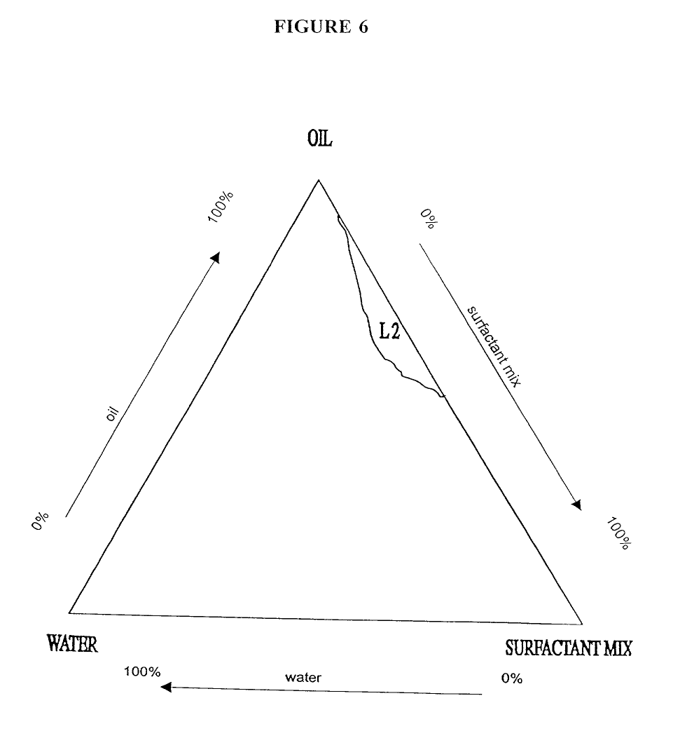

The pseudo-ternary phase diagrams of the three systems investigated are shown in FIGS. 4-6. In particular, the 1:1 Span 80 to Tween 80 mixture is shown in FIG. 4, the 2:1 Span 80 to Tween 80 mixture is shown in FIG. 5, and the 3:1 Span 80 to Tween 80 mixture is shown in FIG. 6. Optically isotropic regions at low water content are designated as L₂ in the Figures. No attempts were made to identify regions at high water and low oil content that were optically isotropic in nature. Unmarked areas in the figures indicate multiphase regions. It is seen that as the Span 80:Tween 80 ratio is increased from 1:1 to 3:1 the region along the surfactant/water axis is significantly affected. Thus, at a 1:1 ratio, it was possible to obtain a mixture containing 18% water and 85% surfactant mix that is optically clear and isotropic. The amount of water that could be included with a 2:1 ratio of Span 80:Tween 80 was reduced to around 5% water and 95% surfactant mixture. Dye solubility tests indicated that all isotropic mixtures along the surfactant/water axis were water-external systems (i.e. normal micellar dispersions). The region of clarity along the surfactant/water axis is further diminished when a 3:1 Span 80:Tween 80 ratio is used.

Mixtures of surfactants with olive oil were optically clear for both the 2:1 and 3:1 ratios of Span 80:Tween 80 along the entire surfactant/oil axis. For the 1:1 ratio, the mixtures were hazy along this axis indicating that Tween 80 solubility in olive oil has been exceeded even at very high oil to surfactant volume ratios. The presence of as little as 0.1% water transformed these hazy mixtures into clear systems.

In general, the isotropic regions tended to narrow down with increasing Span 80:Tween 80 ratios in the oil-poor part of the pseudo-ternary diagram. Thus, it was possible to incorporate over 10% by volume of the aqueous phase with 1:1 ratios at olive oil content around 30%. This is reduced to around 3.5% with a 2:1 ratio of Span 80:Tween 80 containing 30% olive oil. For a 3:1 system, it was not possible to obtain optically clear systems containing even small amounts of water unless the surfactant to oil ratio was close to unity. This is indicative of the incompatibility of the more hydrophobic Span 80 with water in oil-poor regions.

For both the 1:1 and 2:1 systems, the phase boundary appears to be almost a straight line connecting the limit on the surfactant/water axis with the limit on the surfactant/oil axis. For the 3:1 system, however, the isotropic regions appear to be distributed symmetrically along the surfactant/oil axis between 50% oil and 85% oil and exhibiting an apex water solubilization capacity of 5% (volume) at 65% oil. Dye tests with a variety of formulations from oil-poor and oil-rich regions indicated the inability of the water-soluble dye to diffuse freely into the mixtures. This observation suggested that all of these formulations, even those containing only around 30% olive oil, had olive oil as the external phase and could be termed water-in-oil nanoemulsions. The particle sizes of the formulations ranged from around 20 nm to 25 nm in diameter.

### Example 5

### Marker—Nanoemulsion Formulation Skin Permeation Assays

This example describes various marker-nanoemulsion formulation skin permeation assays. In particular, this example describes permeation assays involving the topical administration of inulin-nanoemulsion formulations to the skin of various animal models and permeation assays involving the topical administration of inulin-nanoemulsions to murine skin with various concentrations of inulin. This example also describes a comparison of permeation profiles for two different marker-nanoemulsion formulations (inulin and tranexamic acid nanoemulsion formulations).

A. Preparation of Inulin and Tranexamic Acid Formulations

A nanoemulsion constituting 21.5% Span 80, 10.8% Tween 80, 64.5%, and 3.2% distilled and de-ionized water was pipetted into a scintillation vial and mixed with an aqueous solution of inulin or tranexamic acid containing trace amount of methoxy-³H-inulin (specific activity 159 mCi/g) or methylamine-¹⁴C-tranexamic acid (specific activity 54 mCi/mmol) respectively (both from Amersham Life Sciences, Inc., Arlington Heights, Ill.), and vortexed for 1 minute to obtain clear isotropic systems. The concentrations of inulin and tranexamic acid used were 5 μg/ml and 3 μg/ml respectively. In a few cases, inulin concentrations of 0.75 mg/ml and 1.2 mg/ml were also examined. All marker-nanoemulsions were stored at ambient temperature in tightly capped scintillation vials until used.

B. Nanoemulsion Formulation Application to Murine Skin

Permeation assay were carried using standard skin permeation assays (See, Joel L. Zatz, Skin Permeation: fundamental and application, Allured Pub. Co., Wheaton, Ill., 1993, Chapter 4 ‘In vitro Methods for Measuring Skin Permeation’, pgs 93-111). Briefly, male hairy rats (Sprague Dawley, 8-12 weeks old) or male hairy mice (ICR, 8 weeks old) or male hairless mice (Skh-hr-l, 8-12 weeks old) were sacrificed by a lethal dose (200 mg/kg) intraperitoneam injection of sodium pentobarbital. For hairy rat or mouse studies, the hair was clipped with animal clippers (Oster A5). Full thickness dorsal skin was carefully excised and subcutaneous fat was removed with a dull scalpel. Appropriate sized pieces of skin were then mounted on Franz diffusion cells with a surface area of 1.77 cm² and a receiver capacity of 8 ml (Crown Glass, Somerville, N.J.). The epidermal side of the skin was exposed to ambient conditions while the dermal side was bathed by 0.05 M isotonic HEPES buffer, pH 7.4. The receiver solution was stirred continuously using a small Teflon-covered magnet. Care was exercised to remove any air bubbles between the underside of the skin and the receiver solution. The temperature of the receiver solution was maintained at 37° C.

Following the mounting of the skin, 200 μl or 400 μl of the test inulin formulations were applied to the epidermal surface of the skin are carefully spread to achieve complete surface coverage. A minimum of three cells using skin from at least three different animals was used. All assays were carried out under non-occluded conditions. In the pretreatment assays, the skin mounted on the cells was pre-treated with 200 μl of olive oil for 2 hours. At 2 hours, the skin was washed four times with 2 ml of distilled water and then dabbed dry before application of the desired test formulation. 2 ml aliquots of the receiver solution were withdrawn at predetermined times in order to monitor the kinetics of marker transport across skin. In these experiments, percutaneous absorption was monitored for a total period of 24-48 hours. The receiver solutions were then assayed for radiolabeled markers using a scintillation counter after addition of 15 ml of Ecolite⁺ (ICN Biomedicals, Inc., Irvine, Calif.) to each system.

C. Inulin-Nanoemulsion Permeation in Animal Models

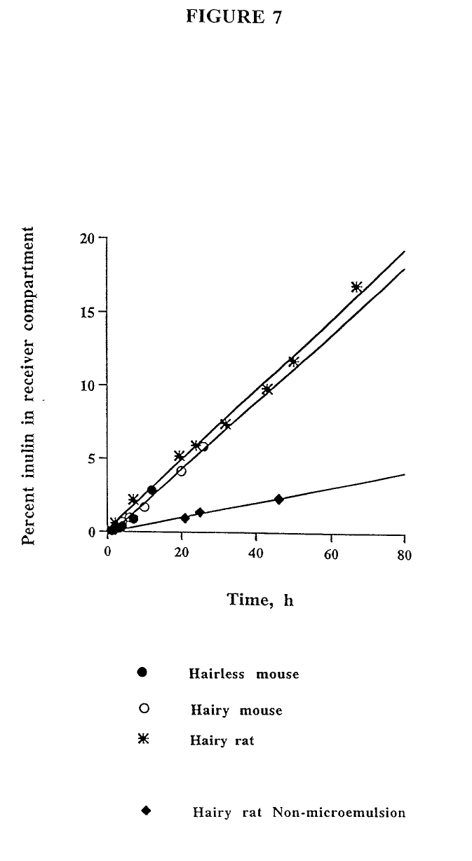

The results of permeation of inulin across hairy and hairless mouse and hairy rat skin following topical in vitro application of an inulin-nanoemulsion formulations (2:1 nanoemulsion formulation, with 5 μg/ml inulin) are plotted in FIG. 7. Standard errors are not shown in FIG. 7, but were typically less than±30%. The plots show the percent of applied nanoemulsion formulation found in the receiver compartment as a function of time. The plots are linear up to at least 24 hours for all animal models with excellent linear regression coefficients (r²≧0.99). The slopes for hairless mouse (0.144% per hour per square centimeter) and hairy mouse (0.135% per hour per square centimeter). The similarity in permeation kinetics or flux for the three animal models suggests that the transport of the water-soluble inulin markers may occur via a common pathway.

FIG. 7 also shows the permeation of inulin from a destabilized nanoemulsion (non-emulsion). Destabilization was induced by adding an equal volume of 5 μg/ml aqueous inulin solution to the 5 μg/ml 2:1 nanoemulsion formulation and vigorously mixing the two in the donor compartment. The permeation rate with the destabilized non-nanoemulsion formulation was dramatically lower (0.03% per hour per square centimeter) than the other nanoemulsion formulations presented in FIG. 7.

D. Effect of Inulin Concentration on Skin Permeation

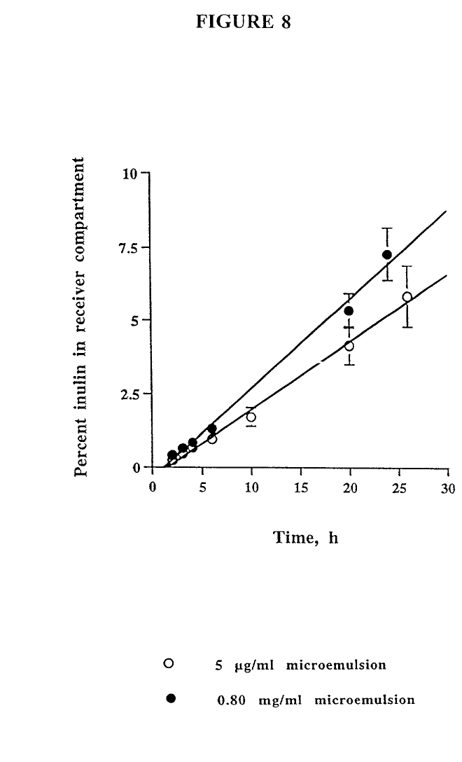

The permeation profiles of inulin across hairy mouse skin following topical in vitro application of 2:1 nanoemulsion formulations with widely differing concentrations of inulin (0.80 mg/ml and 5 μg/ml) are shown in FIG. 8. The plots are linear up to a period of around 20 hours, although a slight curvature is evident at longer time periods in the high inulin concentration profile. The slope from the higher inulin (0.80 mg/ml) nanoemulsion, 0.172% per hour per square centimeter, is slightly higher than the value of 0.116% per hour per square centimeter obtained with the lower (5 μg/ml) system. The similarity of the slopes suggests that the percent of formulation applied that traverses the skin is independent of inulin concentration within the aqueous interior of the nanoemulsion.

E. Comparison of Permeation Profiles for Two Different Markers

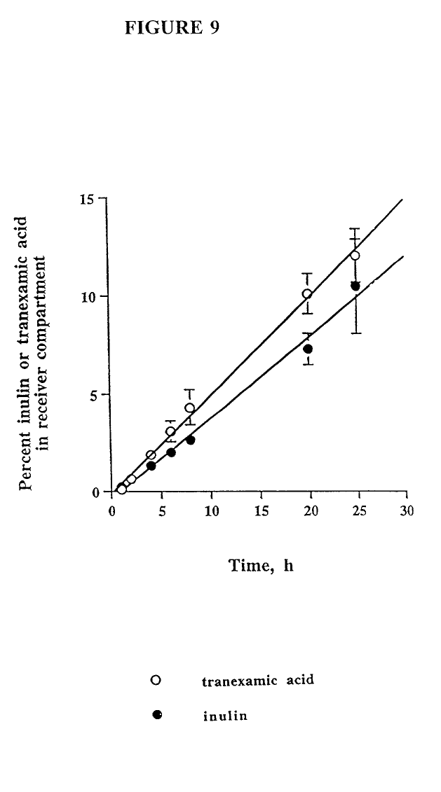

The permeation profiles of nanoemulsion formulations with two different markers were compared. In particular, 2:1 nanoemulsion formulations with 5 μg/ml inulin (MW=5,000 Da) or 3 μg/ml tranexamic acid (MW=157.2 Da) and 0.05 M HEPES buffer, pH 7.4 for the aqueous phase (instead of distilled water) were prepared and compared in hairy mouse skin permeation assays. The results are shown in FIG. 9. The slopes of the plots for the two markers are not very different; 0.283% per hour per square centimeter for tranexamic acid and 0.233% per hour per square centimeter for inulin. Tranexamic acid appears to be transported at a rate roughly 1.2 times faster than inulin across hairy mouse skin. The closeness of the slopes for the two markers indicates that transport of the water-soluble markers from the nanoemulsion formulations is independent of marker molecular weight.

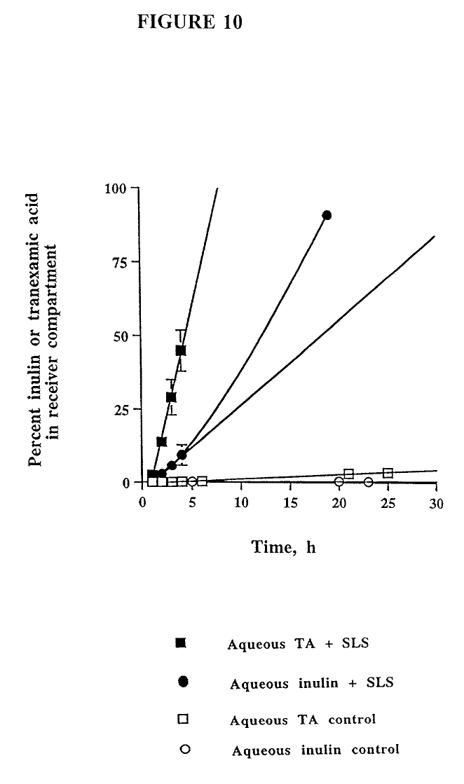

The permeation of these two markers was also examined with 5% sodium lauryl sulfate (SLS), a potent permeation enhancer, or with water. The permeation rates of these two markers in these solutions, unlike the nanoemulsion formulations, is dramatically different. The profiles shown in FIG. 10 indicate a 5-fold difference in permeation rates between tranexamic acid in SLS and inulin in SLA, and a 10 to 15-fold difference in permeation rates between tranexamic acid in water and inulin in water. It is remarkable, therefore, that the two markers, with dramatically different sizes, are transported by the nanoemulsion formulations at very similar rates even though there is a large difference in molecular weight.

### Example 6

### Characterization of Set 1 Nanoemulsion Formulations

This example describes the characterization of various nanoemulsion formulations. In particular, select mixtures within the isotropic region defined in the pseudo-ternary diagrams (See, Example 4 and FIGS. 4-6), were assayed for their physicochemical characteristics as well as their ability to facilitate the transport of water-soluble markers across skin. The systems chosen were well within the isotropic regions defined in the phase diagrams. Three systems (‘Set 1’), one from each ratio of surfactant mixtures (1:1, 2:1, and 3:1), were chosen to represent oil-rich regions so as to maximize their compatibility with sebum. Additionally, the total surfactant concentrations in these oil mixtures was chosen such that they were not dramatically different from each other (range from 25 to 32% by volume). A few systems containing no oil were also selected to determine in the nature of the isotropic mixture has any effects on transport characteristics across skin. The systems selected, along with the compositions, are shown in Table 2.

TABLE 3
Various Emulsion Systems Examined

| | Span | Tween | Olive | Aqu- | | |
|---|---|---|---|---|---|---|
| System | 80 | 80 | Oil | eous | Perm. rt | Corr. rt |
| 1:1 micelle | 42.5% | 42.5% | — | 15.0% | 0.076%/h | 0.041 |
| 2:1 micelle | 64.5% | 32.3% | — | 3.2% | 0.073%/h | 0.038 |
| 1:1 oil-poor | 30.0% | 30.0% | 30.0% | 10.0% | N/D | N/D |
| 1:1 nano | 15.1% | 15.1% | 66.6% | 3.2% | 0.176%/h | 0.141 |
| 2:1 nano | 21.5% | 10.8% | 64.5% | 3.2% | 0.321%/h | 0.286 |
| 3:1 nano | 18.7% | 6.2% | 72.8% | 2.3% | 0.519%/h | 0.484 |
| Aq. Control | — | — | — | 100% | 0.035%/h | — |

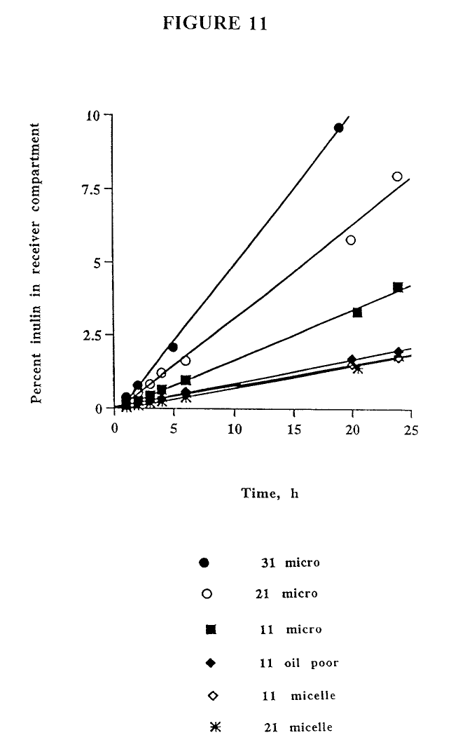

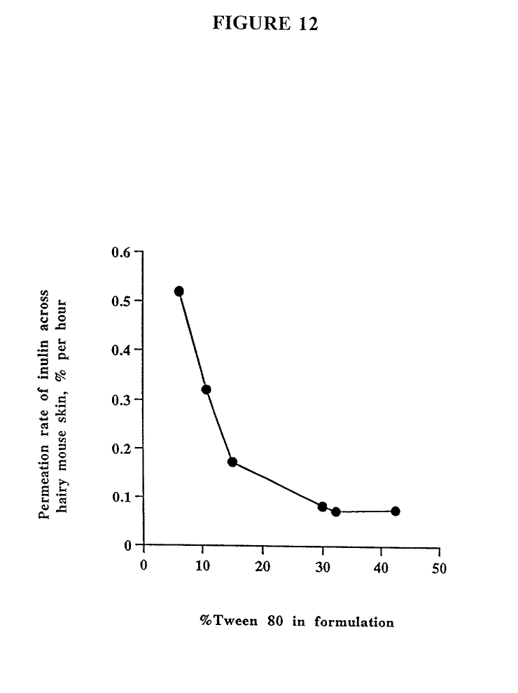

The ability of the various nanoemulsion systems (listed in Table 2) to deliver a marker (inulin) across hairy murine skin was assayed. The processing of murine skin and preparation of inulin containing emulsions was carried out as described in Example 5. FIG. 11 presents profiles of inulin permeation across the hairy murine skin following topical application of the various nanoemulsion systems. The results indicate that higher Span 80:Tween 80 ratios facilitate more rapid transport of inulin from nanoemulsion formulations. The results also indicate that even though Span 80 is more hydrophobic than Tween 80 (and may therefore be expected to have a greater effect on the permeability of the stratum corneum) there is a lack of correlation between permeation rates and Span 80 content. The results also make it clear that permeation rates do not correlate with total surfactant content in the nanoemulsion formulations. On the contrary, there appears to be an inverse correlation between permeation rates and Tween 80 content of the formulations (plotted in FIG. 12).

### Example 7

### Various Nanoemulsion Formulations (Sets 2 and 3)

This example describes the characterization of various nanoemulsion formulations. In particular, the permeation rate of nanoemulsion formulations with Span 80 to Tween 80 ratios of 1:1, 2:1, and 3:1, but with identical total surfactant mixture component (30%) and identical aqueous component (2.7%), were compared (‘Set 2’). A second set of 1:1, 2:1, and 3:1 nanoemulsions was also prepared using a 50/50 (v/v) propylene glycol/isotonic HEPES buffer, pH 7.4, as the aqueous phase was also prepared (‘Set 3’). These three formulations (labelled PG/aq in Table 4) contained 31.7% surfactant mixture component, 62.5% oil component, and 5.8% PG/buffer aqueous component. Also examined as controls were surfactant mixture/aqueous formulations, as well as aqueous solutions of inulin at the same concentrations as in the nanoemulsions. All compositions contained labelled inulin at 5 g/ml. All the nanoemulsions and controls were examined in murine skin permeation assays as described above (See, e.g. Example 3).

Table 4 shows the permeation rates of inulin as well as the corrected rates. The corrected rate is a normalization procedure that was adopted in order to account for differences in the permeation rates of aqueous controls the results reported above in Table 3 and the results reported in Table 4. The corrected rate is simply the difference between the rate for a given formulation and the aqueous control.

TABLE 4
Various Emulsion Systems Examined

| System | Span 80 | Tween 80 | Olive Oil | Aqueous | Perm. rt | Corr. rt |
|---|---|---|---|---|---|---|
| Aq. Control | — | — | — | 100% | 0.192%/h | — |
| 3:1 Nano | 22.5% | 7.5% | 67.3% | 2.7% | 0.870%/h | 0.678 |
| 2:1 Nano | 20.0% | 10.0% | 67.3% | 2.7%% | 0.616%/h | 0.424 |
| 1:1 Nano | 15.0% | 15.0% | 67.3% | 2.7% | 0.447%/h | 0.255 |
| 3:1 PG/aq | 23.8% | 7.9% | 62.5% | 5.8% | 0.823%/h | 0.631 |
| 2:1 PG/aq | 21.1% | 10.6% | 62.5% | 5.8% | 0.615%/h | 0.423 |
| 1:1 PG/aq | 15.8% | 15.8% | 62.5% | 5.8% | 0.357%/h | 0.165 |
| 3:1 surf/aq | 22.5% | 7.5% | — | 60% | 0.154%/h | — |
| 2:1 surf/aq | 20.0% | 10.0% | — | 60% | 0.218%/h | — |
| 1:1 surf/aq | 15.0% | 15.0% | — | 60% | 0.113%/h | — |

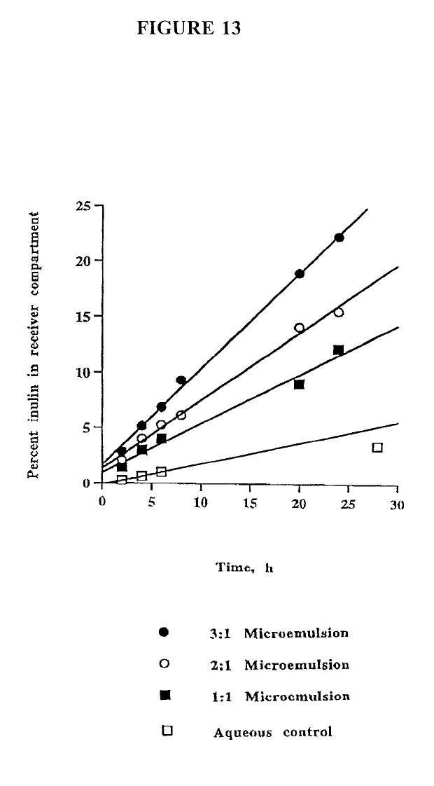

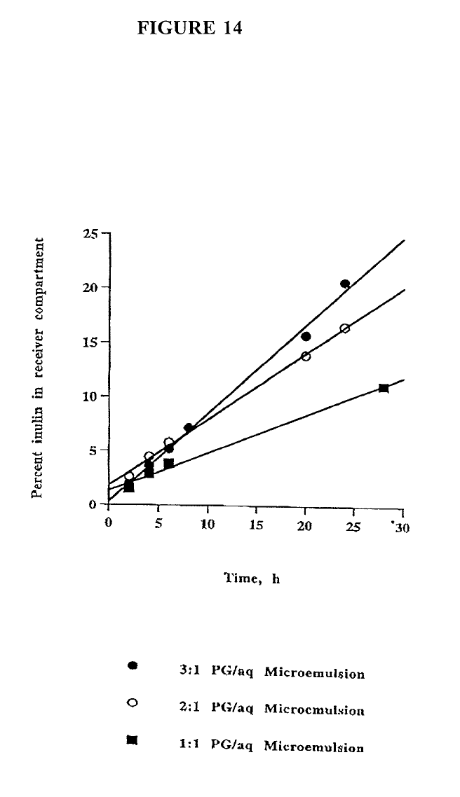

The permeation profiles for the different nanoemulsion formulations are shown in FIGS. 13 and 14. FIG. 13 is a comparison of the profiles from nanoemulsion formulations of identical total surfactant mixture component, oil component, and aqueous component (which was isotonic HEPES buffer, pH 7.4). FIG. 14 shows a comparison of profiles from nanoemulsion formulations, also with the same amount of each component, but with an aqueous phases containing 50/50 (v/v) mixture of PG and isotonic HEPES buffer, pH 7.4. These results, along with those presented in Table 4, demonstrate that the inclusion of 50% by volume of propylene glycol in the aqueous component allows a higher degree of incorporation of the aqueous component into the nanoemulsion formulation (arpox. double). Furthermore, the permeation rates between set 2 nanoemulsions (without the propylene glycol), and set 3 nanoemulsions (with propylene glycol) was not significantly different.

The results of permeation of inulin from an aqueous solution containing only the surfactant mixtures (surf/aq) indicates that these nonionic surfactants provide a negligible degree of enhancer action. Indeed, the permeation rates from these systems are not dramatically different from those obtained with aqueous controls.

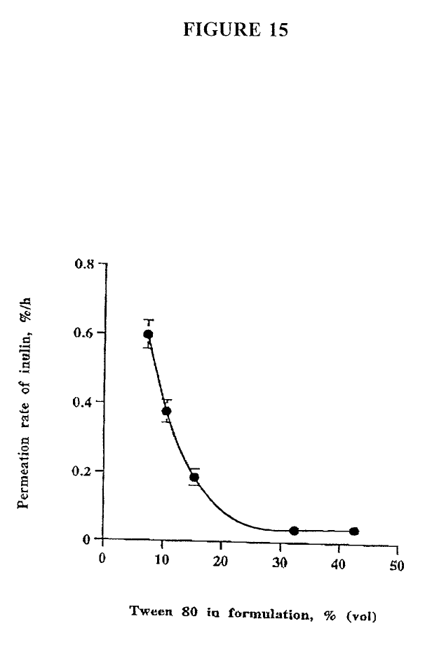

Table 5 shows a summary of the corrected permeation rates of inulin across hairy mouse skin from these two sets of nanoemulsions (i.e. Sets 2 and 3), and the set described in Example 6 (i.e. Set 1). These results indicate that nanoemulsion formulations with higher ratios of Span 80 to Tween 80 (e.g. 3:1) transport inulin more efficiently than lower ratios. These average permeation rates of inulin from these nanoemulsion listed in Table 5 are plotted in FIG. 15 as a function of Tween 80 content in the formulation. It is seen that the rates decrease steadily with increasing Tween 80 content until a plateuing effect is observed at high Tween 80 levels.

TABLE 5
Comparison of Inulin Permeation Rates for Different Systems

| | 3:1 systems | 2:1 systems | 1:1 systems |
|---|---|---|---|
| Set 1 Nanoemulsions | 0.484%/h | 0.286%/h | 0.141%/h |
| Set 2 Nanoemulsions | 0.678%/h | 0.424%/h | 0.255%/h |
| Set 3 PG/aq Nanoemulsions | 0.631%/h | 0.423%/h | 0.165%/h |
| average | 0.598%/h | 0.378%/h | 0.187%/h |
| standard deviation | 0.101 | 0.079 | 0.060 |

### Example 8

### Treatment of AIDS Associated Kaposi's Sarcoma

This example describes the treatments of a patient with AIDS associated Kaposi's Sarcoma. Kaposi's sarcoma (KS) is a proliferative disease of vascular origin frequently associated with Human Immunodeficiency Virus-1 (HIV-1) infection (Havercos et al. N. Engl. J. Med 312:1518 [1985]). KS typically occurs as lesions in the skin, although, in most AIDS-KS patients, visceral lesions are also present. KS often arises as multiple disseminated skin lesions that in early stages resemble benign capillary hemangiomas or vascularized chronic inflammatory foci. In more advanced stages, the lesions appear as multiple purplish to brown subcutaneous plaques or nodules, often with a verrucose surface. Other KS histological features are extravascular hemorrhage with hemosiderin deposition, anaplastic fibroblast-like proliferation, and a granulation-like inflammatory reaction. Robbins et al. “Basic Pathology” p. 286 (W. B. Saunders Co., 2d ed. 1976).

In this example, the lesions of a patient with AIDS associated KS are treated topically with a nanoemulsion formulation (3:1 Span 80-Tween 80) comprising huINFα2 expression plasmids at a concentration of 0.2 mg/ml (See, e.g. Example 1 above). Treatment is continued for 5 weeks with daily topical application of the nanoemulsion formulation comprising huINFα2 expression plasmids.

### Example 9

### Treatment of Systemic Lupus Erythematosus Skin Lesions

This example describes the treatment of the skin lesions normally associated with systemic lupus erythematosus (SLE). SLE is a chronic autoimmune disease of unknown etiology. Skin lesions are a major component of the pathophysiological manifestations that characterize SLE. Among the skin lesions that characterize SLE are discoid lupus erythematosus (DLE) and subacute cutaneous lupus erythematosus (SCLE). Both of these skin lesions may occur in patients without additional evidence of SLE, or may complicate the clinical course of SLE. Five histological abnormalities are generally associated with DLE including: hyperkeratosis with keratotic follicular plugging; thinning and flattening of the stratum malpighii; hydropic degeneration of basal cells; a lymphoid cell infiltrate involving the follicles and other appendages; and edema, vasodilation and erythrocytic extravasation into the upper dermis. DLE is primarily an epidermal lesion with significant involvement of the follicular and perifollicular structures. In SCLE, hydropic degeneration of the basal cells and dermal edema are more severe, whereas hyperkeratosis and lymphocytic infiltrates are less severe. Both SCLE and DLE have the potential to be disfiguring lesions with high incidence of hypopigmentation and scarring.

In this example, the lesions of a patient with SLE are treated topically with a nanoemulsion formulation (3:1 Span 80-Tween 80) comprising huINFα2 expression plasmids. (See, e.g. Example 1 above). Initially, the patient is administered a test dose of the nanoemulsion formulation with a plasmid concentration of 0.2 mg/ml in a single unit dose of 100 ml (i.e. 20 mg of plasmid are administered). The test dose is applied to the skin lesions of the patient to be treated and gently rubbed with a gloved finger to distribute the material across the entire surface area to be treated. The area is then covered with an occlusive petrolatum dressing. Following this topical application, the patient is observed for 2 hours for any local sensitivity, dermal reactions or systemic reactions.

Absent adverse effects, the patient is then treated with the nanoemulsion formulation in a similar manner twice daily according to the following schedule: 0.16 mg/ml plasmid concentration for 4 days; 0.48 mg/ml plasmid concentration for 12 days; 1.60 mg/ml plasmid concentration for 40 days; and 4.80 mg/ml plasmid concentration for 120 days. Treatment is terminated early if complete resolution of skin lesions is achieved prior to completing the entire treatment schedule.

### Example 10

### Nanoemulsion Preparation

This example describes a general procedure employed to generate a nanoemulsion preparation containing a plasmid. In particular, this example describes a method used to prepare small amounts of a nanoemulsion where an aqueous phase containing a plasmid is suspended in a lipid phase containing cosurfactants. A general step by step procedure is described below.

In order to mix the cosurfactants in the appropriate ratio, the following steps may be employed. First, obtain or empirically determine the densities of each of the cosurfactants. Calculate the volume of each cosurfactant required based on the desired cosurfactant volume ratio and desired volume (e.g. if a 2:1 volume ratio of Span 80:Tween 80 is desired with a total cosurfactant volume of 60 mL, 40 mL of Span 80 and 20 mL of Tween 80 are required). Using the densities of the cosurfactants, calculate the weight of each cosurfactant required (as the cosurfactants are often very viscous, the amount used is preferably confirmed by weight). Next, place a container (e.g. conical tube or solution bottle), on a scale without a cap. Tare the container on the scale. Then, using a transfer means (e.g. positive-displacement pipette), transfer the appropriate volume and weight of each cosurfactant into the container. Finally, cap and vortex the container until the contents are thoroughly mixed (e.g. 1-2 minutes). The cosurfactants should be completely miscible.

In order to mix the cosurfactants with the primary lipid, the following steps may be employed. First, obtain or empirically determine the densities of the cosurfactant mixture and of the primary lipid. Calculate the volume of the cosurfactant mixture and primary lipid required based on the desired volume ratio and volume (e.g., if a 30:70 volume ratio of cosurfactants to olive oil is desired with a total volume of 50 mL, 15 mL of the cosurfactant mixture and 35 mL of olive oil are used). Using the densities of the cosurfactant mixture and primary lipid, calculate the weight of each component that is needed (as the cosurfactants and lipids are often very viscous, the amount used is preferably confirmed by weight). Next, place a container (e.g. conical tube or solution bottle) on a scale without a cap. Tare the container on the scale. Then, using a transfer means (e.g. a positive-displacement pipette) transfer the appropriate volume and weight of the cosurfactant mixture and primary lipid into the container. Finally, cap and vortex the container until the contents are thoroughly mixed (e.g. 1-2 minutes). The cosurfactants and the primary lipid should be completely miscible.

The lipid phase may be filtered by employing the following steps. First, take the complete lipid phase to a laminar flow hood. Attach a filter unit to the house vacuum, and include an inline filter to prevent aerosols from entering the vacuum system. With the vacuum level at approximately half-way, carefully transfer (e.g. pipette or pour) the lipid phase into the upper compartment of the filter unit. Next, observe the lipid phase to ensure that it is passing through the filter. The filtration process can take several hours with a highly viscous lipid phase. Check the filter unit integrity periodically. If the filter becomes clogged, switch the remaining unfiltered lipid phase to a new unit. Finally, when all of the lipid phase has been filtered, cap the collection reservoir from the filter unit and label the container with the contents and date.

The aqueous phase containing a plasmid may be prepared employing the following steps. First, determine the concentration of the plasmid in the aqueous phase (e.g. an aqueous phase of Tris-EDTA). The aqueous phase may be sterilized if desired (sterilization may decrease plasmid concentration in the aqueous phase). If additional diluent is required, this may also be filter sterilized. Adjust the concentration to the desired level using additional sterile diluent.

The aqueous phase may be added to the lipid phase by employing the following steps. First, obtain or empirically determine the densities of the lipid phase and aqueous phase. Calculate the volume of each phase required based on the desired phase volume percentage and desired total volume (e.g., if a 3% volume percentage of aqueous phase in lipid phase is desired with a total volume of 10 mL, 300 μL of aqueous phase and 9700 μL of lipid phase are required.) Then, using the densities of the cosurfactants, calculate the weight of each phase required (as the lipid phase is often very viscous, the amount used is preferably confirmed by weight). Next, place a container (e.g. conical tube or solution bottle) on a scale without a cap. Tare the container on the scale. Using a transfer means (e.g. a positive-displacement pipette) transfer the appropriate volume and weight of each phase into the container. Finally, cap the container and swirl it to mix.

The solution will initially be cloudy, but will clear as the nanoemulsion forms. If after 5 minutes, the solution is still cloudy, place it on a tube rotator for 30 minutes. If the solution still does not clear, slowly add small amounts of the lipid phase to the solution, carefully recording the volumes and weights added. After each addition, repeat the mixing process. Once the solution has cleared, calculate the new phase volume ratio present in the final solution. The nanoemulsion is now ready for use. The nanoemulsion aspect of the solution should be stable at room temperature for about 6 months, although the contents of the aqueous phase may not be stable at room temperature for that long.

### Example 11

### Topical Transfection of Luciferase Plasmid in Nanoemulsion

This example describes the transfection of murine skin with a nanoemulsion preparation containing a luciferase plasmid, and in vivo imaging of luciferase expression in the murine skin. The plasmid employed in this example was the pRET2-Luc plasmid, which contains the MPSV promoter region and firefly (Photinus pyralis) luciferase gene. This plasmid was purified using a modified alkaline lysis procedure with the CONCERT plasmid purification system (GibcoBRL, Gaithersburg Md.) according to manufacturer's instructions. The plasmid was further purified via cesium chloride ultracentrifugation. The highly purified plasmid DNA was assayed for endotoxin via a limulus amboebocyte lysate (LAL; Biowhittaker, Walkersville Md.) assay prior to nanoemulsion preparation.

The nanoemulsion preparation containing the pRET2-Luc plasmid was prepared generally according to the procedure described above in Example 10. A 2:1 volume ratio of Span80:Tween 80 was employed, as well as a 9:4 volume ratio of cosurfacants to olive oil. The aqueous phase was Tris-EDTA (pH 7.4, Sigma), and was not filter sterilized. Prior to concentration adjustment, the aqueous phase contained the Luciferase plasmid at 6.495 mg/mL. After concentration adjustment, the aqueous phase contained Luciferase plasmid at approximately 5.0 mg/ml. The aqueous phase in lipid phase concentration was 3.47% in the final nanoemulsion preparation.

This nanoemulsion was used to transform mouse skin. All animal protocols were approved by the University of Michigan Committee on the Use and Care of Animals and performed according to institutional guidelines. Female B6.MRL-Fas 1 pr mice (9-10 weeks of age) were obtained from Jackson Laboratories (Bar Harbor Me.) and individually housed under specific pathogen free conditions in the Unit for Laboratory Animal Medicine. Mice were individually housed to prevent cross contamination and premature removal of dressings during experimental procedures and were identified by ear punches.

Individual animals were briefly anesthetized using isoflurane. A plastic cylinder (0.95 cm) was used as a template to mark the center of the shaved dorsal area with red ink where the nanoemulsion test substance was to be applied. Fifty (50) ml of the nanoemulsion was pipetted onto the center of the treatment area, or marked region of the murine skin using a positive displacement pipette. The nanoemulsion test substance was applied using a circular motion with a clean, gloved finger only to the treatment area identified on the back of the anesthetized mouse. A flexible adhesive bandage was applied to the treatment area approximately 1-2 min after the nanoemulsion test substance was rubbed onto the dorsal skin. The mouse was then returned to its cage. The bandage remained on the mouse, covering the treated area, for approximately one hour. Nanoemulsion application procedures were repeated on a daily basis for 4 days and mice imaged on the 5th day. The overall macroscopic appearance of the treated area and surrounding tissue was visually evaluated daily.

Anesthetized mice were imaged using the IVIS Molecular Imaging System and Living Image software (Xenogen, Alameda Calif.) in the University of Michigan Imaging Core (Department of Radiology). Imaging was started approximately 1-2 minutes after application of 50 ul of the luciferin solution (Xenogen; 40mg/ml in phosphate buffered saline) in nanoemulsion. Each image was acquired for a 3 minute time period. Numerical photon data count, as well as images which show emitted light as a pseudocolor graphic, overlayed with a photo image of the mouse were obtained. The results show that the skin of the mice treated with expression plasmid DNA, followed by topical application of luciferin substrate, exhibited emission of light from the surface of the skin. The area of light emission was confined to the area treated with nanoemulsion containing expression plasmid DNA. Controls treated with an aqueous solution of expression plasmid DNA had no detectable light emission from the skin.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in medicine, chemistry, and molecular biology or related fields are intended to be within the scope of the following claims.

We claim:

1. A composition comprising a nanoemulsion formulation, wherein the nanoemulsion formulation comprises an aqueous component, an oil component, and a surfactant mixture component, wherein said surfactant mixture component comprises a low HLB value surfactant and a high HLB value surfactant.

2. The composition of claim 1, wherein the ratio of said low HLB value surfactant to said high HLB value surfactant is at least 2:1.

3. The composition of claim 1, wherein the ratio of said low HLB value surfactant to said high HLB value surfactant is at least 3:1.

4. The composition of claim 1, wherein said low HLB value surfactant has an HLB value between approximately 3.3 and 5.3 and the high HLB value surfactant has an HLB value between approximately 14.0 and 16.0.

5. The composition of claim 1, wherein said nanoemulsion formulation further comprises a biological agent.

6. The composition of claim 1, wherein said nanoemulsion does not contain short-chain alcohols.

7. The composition of claim 1, wherein said low HLB value surfactant is present in a greater amount than said high HLB value surfactant.

8. The composition of claim 1, wherein said surfactant mixture component comprises a low HLB value non-ionic surfactant and a high HLB value non-ionic surfactant.

9. A composition comprising a nanoemulsion formulation that permits a skin permeation rate of at least 0.447% per hour for a biological agent in said nanoemulsion formulation.

10. The composition of claim 9, wherein said skin permeation rate is selected from at least 0.519% per hour, at least 0.615% per hour, and at least 0.823% per hour.

11. A composition comprising a nanoemulsion formulation that permits an expression vector to express a recombinant peptide at a mean level of at least 57.0 pg/cm² in cells.

12. The composition of claim 11, wherein said recombinant peptide is expressed at a mean level selected from at least 100.0 pg/cm², at least 285.0 pg/cm², and at least 376.0 pg/cm².

13. A composition comprising a nanoemulsion formulation that permits an expression vector to express RNA transcripts at a level of at least 5.0×10⁴ transcripts/cm² in cells.

14. A method comprising;
a) providing;
i) a nanoemulsion formulation comprising an aqueous component, an oil component, a biological agent, and a surfactant mixture component, wherein said surfactant mixture component comprises a low HLB value surfactant and a high HLB value surfactant, and
ii) a subject; and
b) administering said nanoemulsion formulation to said subject.

15. The method of claim 14, wherein said nanoemulsion is administered non-parenterally to said subject.

16. The method of claim 14, wherein said biological agent is selected from the group consisting of drugs, nucleic acids, expression vectors, and peptides.

17. The method of claim 14, wherein said biological agent comprises an expression vector.

18. The method of claim 17, wherein said subject comprises skin cells and said administering comprises administering said nanoemulsion formulation to said skin cells of said subject such that said expression vector expresses RNA transcripts at a level of at least 5.0×10⁴ transcripts/cm² in said skin cells of said subject.

19. The method of claim 18, wherein said RNA transcripts are antisense RNA.

20. The method of claim 14, wherein said subject further comprises skin and said nanoemulsion formulation permits a skin permeation rate of at least 0.447% per hour for said biological agent in said skin of said subject.
